(19) United States
(12) Patent Application Publication
(10) Pub. No.: US 2026/0207745 A1
(43) Pub. Date: Jul. 23, 2026

(54) COMPOSITIONS AND METHODS OF USE THEREOF FOR THE TREATMENT OF VIRALLY DRIVEN CANCERS

(71) Applicant: YALE UNIVERSITY, New Haven, CT (US)

(72) Inventors: Jeffrey ISHIZUKA, Woodbridge, CT (US); Alexander FREY, New Haven, CT (US); Kelly OLINO, Guilford, CT (US)

(21) Appl. No.: 19/138,692

(22) PCT Filed: Dec. 14, 2023
(86) PCT No.: PCT/US2023/084086

§ 371 Date: Jun. 13, 2025

Related U.S. Application Data
(60) Provisional application No. 63/387,439, filed on Dec. 14, 2022.

(51) Int. Cl. A61K 40/11; A61K 31/7105; A61K 35/15; A61K 35/17; A61K 39/00; A61K 39/12; A61K 40/19; A61K 40/32; A61K 40/42; C07K 14/725; C12N 5/0783; C12N 5/0784; C12N 15/11

(52) U.S. Cl. CPC A61K 40/11; A61K 31/7105; A61K 35/15; A61K 35/17; A61K 39/12; A61K 40/19; A61K 40/32; A61K 40/42; C07K 14/7051; C12N 5/0636; C12N 5/0639; C12N 15/11; A61K 2039/53; C07K 2317/56; C12N 2830/34

(57) ABSTRACT

Immunogenic compositions and methods of use thereof, for eliciting an immune response against one or more virally driven cancers are described. The compositions typically include one or more viral antigens expressed in the virally driven cancers, or a nucleic acid encoding the same, and optionally one or more adjuvants. Preferably, the compositions include nucleic acids (e.g., mRNA) encoding one or more viral antigens. Exemplary antigens include those derived from a truncated form of the viral Large T Antigen (LTA) or Small T Antigen (STA) of Merkel Cell Polyomavirus (MCPyV), or E2, E5, or E6 proteins of HPV. Pharmaceutical compositions and dosages including one or more of the compositions and an excipient, and methods of immunizing subjects and priming and engineer T cells for use in adoptive therapy are also provided.

### CROSS-REFERENCE TO RELATED APPLICATIONS

This application claims the benefit of and priority to U.S. Provisional Application No. 63/387,439 filed Dec. 14, 2022, which is hereby incorporated by reference in its entirety.

### STATEMENT REGARDING FEDERALLY SPONSORED RESEARCH

This invention was made with government support under K08CA245112-01 awarded by the National Institutes of Health. The government has certain rights in the invention.

### REFERENCE TO SEQUENCE LISTING

The Sequence Listing submitted as a text file named “YU_8466_PCT_ST26.xml” created on Dec. 14, 2023, 2023, and having a size of 48,069 bytes bytes is hereby incorporated by reference pursuant to 37 C.F.R. § 1.834(c)(1).

### FIELD OF THE INVENTION

The invention is generally in the field of immunogenic compositions and methods of use thereof to increase immune responses against virally driven cancers.

### BACKGROUND OF THE INVENTION

Merkel Cell Carcinoma (MCC) is a rare cutaneous malignancy of unclear neuroendocrine origin that is locally aggressive with potential for early metastasis. MCC has a rapidly increasing incidence and affects up to 1 per 100,000 individuals in the United States. It is most common among Caucasians with a 2:1 male to female predominance and increased incidence among the elderly. The risk of developing MCC is also dramatically increased among immunosuppressed individuals, such as transplant recipients and those with preexisting hematologic malignancies (Paulson K G, et al., J Am Acad Dermatol. 2018 March; 78(3):457-463.e2).

The etiology of MCC falls into two categories: ultraviolet light mediated DNA damage in the minority of cases, and association with Merkel Cell Polyomavirus (MCPyV) in the majority of cases. MCPyV was discovered in 2008 and is estimated to be associated with 80% of MCC cases in the United States.

Various therapies have been used for MCC clinic treatments, but the outcome of clinical prognosis is poor, with a low rate of 5-year overall survival and high risk of recurrence owing to immune compromise (Bichakjian, C. K. et al., J. Natl Compr. Canc. Netw. 16, 742-774 (2018), Cornejo, C. & Miller, C. J. Dermatol. Clin. 37, 269-277 (2019)).

It is an object of the invention to provide vaccine and cell compositions for Merkel cell polyomavirus-driven Merkel cell carcinoma and other virally-driven cancer, and methods for making and methods of use thereof.

It is another object of the invention to provide vaccine compositions and methods for making for other virally driven cancers.

### SUMMARY OF THE INVENTION

Immunogenic compositions and methods of use thereof, are provided. The immunogenic compositions and methods are particularly suited for inducing or stimulating a T cell mediated immune response to one or more viral antigens. The immunogenic compositions typically include a nucleic acid, preferably as an mRNA, encoding a viral antigen or an immunogenic fragment thereof, or the antigen expressed therefrom, and optionally an adjuvant. In preferred embodiments, the viral antigens are derived from a virus that causes cancer. Exemplary viral antigens are derived from Merkel Cell Polyomavirus (MCPyV), Hepatitis B virus (HBV), Hepatitis C virus (HCV), Human papilloma virus (HPV), Kaposi's sarcoma-associated herpesvirus, Epstein-Barr virus (EBV), or human T-cell lymphotropic virus. In one embodiment, the viral antigen is derived from MCPyV, for example, the truncated form of the viral Large T Antigen (LTA) or Small T Antigen (STA) of MCPyV. In another embodiment, the viral antigen is derived from an E2, E5, or E6 protein of HPV.

In some embodiments, the viral antigen or an immunogenic fragment thereof includes the amino acid sequence of any one of SEQ ID NOs:1-5 or 25, and a variant thereof at least 70%, 75%, 80%, 85%, 90%, 91%, 92%, 93%, 94%, 95%, 96%, 97%, 98%, or 99% sequence identity thereto. In some embodiments, the nucleic acid includes the nucleotide sequence of any one of SEQ ID NOs:6-10 or 24.

In some embodiments, the nucleic acid further includes a nucleotide sequence encoding a signal peptide. In a preferred embodiment, the signal peptide is derived from a signal peptide of a full-length coronavirus spike protein, for example from a coronavirus variant of SARS-CoV-2 selected from the group consisting of SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant). In one embodiment, the signal peptide has the nucleotide sequence of SEQ ID NO:11.

In further embodiments, the nucleic acid includes a nucleotide sequence encoding an affinity tag such as FLAG-tag having the amino acid sequence DYKDDDDK (SEQ ID NO: 21). For example, in some embodiments, the nucleic acid includes a nucleotide sequence encoding a viral antigen or an immunogenic fragment thereof, a signal peptide at the N-terminus, and an affinity tag at the C-terminus of the viral antigen or an immunogenic fragment thereof.

In some embodiments, the nucleic acid further includes a 5′ untranslated region (UTR) sequence and/or a 3′ UTR sequence. Exemplary 5′ UTR sequences include the nucleotide sequence of any one of SEQ ID NOs:16-19. An exemplary 3′ UTR sequence includes the nucleotide sequence of SEQ ID NO:20. In some embodiments, the nucleic acid further includes a poly(A) tail.

For example, provided is a double stranded DNA sequence including the disclosed immunogenic composition, optionally further including (i) one or more restriction sites, (iii) promoter region, (iv) TRILINK CAP site, and/or (v) traditional KOZAK sequence. An exemplary promoter region is a T7 promoter. Exemplary double stranded DNA sequence has the nucleotide sequence of any one of SEQ ID NOs:6-10 or 25.

Pharmaceutical formulations of the disclosed immunogenic composition, and one or more pharmaceutically acceptable carrier are also provided.

In preferred embodiments, the immunogenic composition is an mRNA.

In some embodiments, the immunogenic composition is encapsulated within and/or associated with a delivery vehicle that increases the serum half-life of the immunogenic composition as compared to the serum half-life of the same amount of the immunogenic composition alone. An exemplary delivery vehicle is a lipid nanoparticle, for example, the lipid nanoparticle formulated with SM-102, 1,2-DSPC, cholesterol, and DMG-PEG. In one embodiment, the lipid nanoparticle is formulated with SM-102, 1,2-DSPC, cholesterol, and DMG-PEG in a lipid molar ratio of 50:10:38.5:1.5. In other embodiments, the pharmaceutical formulation is formulated for intranasal or by intravascular or intramuscular administration.

Methods of eliciting an immune response in a subject in need thereof are also described. Typically, the methods administer an effective amount of the pharmaceutical formulation to elicit an immune response. In some embodiments, the pharmaceutical formulation is administered by intranasally, or by intravascular or intramuscular injection. In some embodiments, the methods administer to a subject at risk of having a viral infection or cancer caused therefrom, for example, a viral infection or cancer caused by one or more of Merkel Cell Polyomavirus (MCPyV), Hepatitis B virus (HBV), Hepatitis C virus (HCV), Human papilloma virus (HPV), Kaposi's sarcoma-associated herpesvirus, Epstein-Barr virus (EBV), and human T-cell lymphotropic virus. Therapeutic and prophylactic applications are provided. In some embodiments, pharmaceutical formulation is administered to a subject with cancer, optionally a virally driven cancer. Thus, in some embodiments, the pharmaceutical formulation is administered to a subject that has been diagnosed with a virally driven cancer. In some embodiments, the pharmaceutical formulation is administered to a subject at risk of developing cancer. In further embodiments, the subject has or had a viral infection that can lead to virally driven cancer. An exemplary virus is MCPyV. In some embodiments, the pharmaceutical formulation is administered to the subject has or is at risk of developing Merkel cell carcinoma, liver cancer, cervical and other anogenital cancers, Burkitt's lymphoma, nasopharyngeal carcinoma, Kaposi's sarcoma, or adult T-cell leukemia. In some embodiments, prophylactic use cases for MCC are administered to selected populations, such as patients with lymphoproliferative disease associated with a higher risk of MCC (e.g. Chronic lymphocytic lymphoma) or patients who otherwise are or are going to be immunosuppressed (e.g. solid organ transplant patients taking immunosuppressive medications). Some methods optionally include a patient selection step. Methods for enriching T cells specific for a viral or tumor antigen are also provided. Generally, the methods involve the steps of optionally i) isolating T cells from a subject; and ii) stimulating the T cells using the viral or tumor antigen or nucleic acid encoding the same, such as those provided herein, optionally one or more culturing steps before and/or after step ii) with or without one or more immunostimulants such as IL-2, IL-7, IL-15, STING agonists, RIG-I etc.

In some embodiments, the viral or tumor antigen for stimulating the T cells are expressed and presented by monocyte-derived dendritic cells. In some embodiments, the monocyte-derived dendritic cells are derived from the same or different subject as the T cells. In some embodiments, the dendritic and/or T cells are isolated from peripheral blood mononuclear cells of the subject. In preferred embodiments, the subject is one with a viral infection that can lead to cancer or a virally driven cancer. In one embodiment, the subject has MCC. In some cases, the method also includes the step of isolating T cells that are specific towards the viral or tumor antigen, sequencing the T cell receptors (TCRs) from the enriched T cells, determining the binding characteristics of the TCRs towards the viral or tumor antigen, and/or selecting TCRs based on desired binding characteristics. In other embodiments, the method includes the step of using one or more of these TCRs with desired binding characteristics for use in T cell therapy such as adoptive transfer of one or more T cells expressing one or more of these TCRs with desired binding characteristics.

Engineered T cell receptor (TCR) are also provided. In some embodiments, the TCRs including an alpha chain variable domain including the amino acid sequence of SEQ ID NO:27, 29, 31, 33, 35, 37, 39, 41, 43, or 45, or a variant thereof with at least 70% sequence identity thereto, and/or a beta chain variable domain including the amino acid sequence of SEQ ID NO:28, 30, 32, 34, 36, 38, 40, 42, 44, or 46, or a variant thereof with at least 70% sequence identity thereto, wherein the TCR is specific for an LTA antigen.

For example, the TCR can include:
an alpha chain variable domain including the amino acid sequence of SEQ ID NO:27 and a beta chain variable domain including the amino acid sequence of SEQ ID NO:28;
an alpha chain variable domain including the amino acid sequence of SEQ ID NO:29 and a beta chain variable domain including the amino acid sequence of SEQ ID NO:30;
an alpha chain variable domain including the amino acid sequence of SEQ ID NO:31 and a beta chain variable domain including the amino acid sequence of SEQ ID NO:32;
an alpha chain variable domain including the amino acid sequence of SEQ ID NO:33 and a beta chain variable domain including the amino acid sequence of SEQ ID NO:34;
an alpha chain variable domain including the amino acid sequence of SEQ ID NO:35 and a beta chain variable domain including the amino acid sequence of SEQ ID NO:36;
an alpha chain variable domain including the amino acid sequence of SEQ ID NO:37 and a beta chain variable domain including the amino acid sequence of SEQ ID NO:38;
an alpha chain variable domain including the amino acid sequence of SEQ ID NO:39 and a beta chain variable domain including the amino acid sequence of SEQ ID NO:40;
an alpha chain variable domain including the amino acid sequence of SEQ ID NO:41 and a beta chain variable domain including the amino acid sequence of SEQ ID NO:42;
an alpha chain variable domain including the amino acid sequence of SEQ ID NO:43 and a beta chain variable domain including the amino acid sequence of SEQ ID NO:44; or
an alpha chain variable domain including the amino acid sequence of SEQ ID NO:45 and a beta chain variable domain including the amino acid sequence of SEQ ID NO:46.

The alpha and beta variable domains can each include three complementarity determination regions: CDR1, CDR2, and CDR3. In some embodiments, the CDR3 of the alpha variable domain is SEQ ID NO:27, 29, 31, 33, 35, 37, 39, 41, 43, or 45, or a variant thereof with at least 70% sequence identity thereto and the CDR3 of the beta variable domain is SEQ ID NO:28, 30, 32, 34, 36, 38, 40, 42, 44, or 46, or a variant thereof with at least 70% sequence identity thereto.

In some embodiments, TCR is a human or humanized TCR.

In some embodiments, the TCR is a soluble TCR, wherein the soluble TCR does not include a transmembrane domain or includes transmembrane domain that is a CD28 transmembrane domain or a CD8a transmembrane domain, or further includes a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing.

In some embodiments, the TCR includes a detectable label, a therapeutic agent, an immunotoxin, or a chemotherapeutic agent.

Nucleic acids such as expression vectors encoding the TCR are also provided. Host cells, such as T cells, engineered to express the TCR, are also provided and can be used in any of the cell therapies, e.g., adoptive T cell therapies, provided herein.

The disclosed immunogenic compositions, cells, and pharmaceutical formulations thereof can be used alone or in combination with other interventions. In some embodiments, the subject has advanced, inoperable cancer and/or metastases. In some embodiments, the immunogenic composition, cells, or pharmaceutical formulation is administered in combination with or second therapeutic intervention such as conventional antiviral and/or anticancer therapeutics, or procedures for example, radiation or surgery. In particular embodiments, the immunogenic compositions, cells, and pharmaceutical formulations is administered to a subject a with a virally driven cancer as an adjunct to surgery or radiation, e.g., before, during, and/or after surgery or radiation. For example, in some embodiments, the subject first receives surgery and/or radiation to remove one or more tumors and subsequently receives the immunogenic compositions, cells, and pharmaceutical formulations to treat remaining tumor cells. Additionally or alternatively, the subject can be administered the immunogenic compositions, cells, and pharmaceutical formulations before surgery or radiation. Such administration may be used to shrink the tumor prior to surgery.

### BRIEF DESCRIPTION OF THE DRAWINGS

FIG. 1 is a schematic diagram of a pUC57 vector.

FIG. 2 is a schematic diagram of expression vector for LTA and STA expression in mouse or human model systems.

FIG. 3 is a line graph showing tumor volume in mm³ over a period of 20 days post challenge in groups of LTA-expressing, wild-type, and empty vector control B16 tumors in wild-type mice.

FIG. 4 is a schematic diagram of LTA vaccine production plasmid map.

FIGS. 5A-5B are graphs showing tumor volume in mm³ over a period of 18 days post challenge (FIG. 5A) and survival over a period of 48 days (FIG. 5B) in groups of LTA-expressing and Empty Vector B16 tumors treated with LTA vaccine at 6 μg or 15 μg or without LTA vaccine (Placebo).

FIGS. 6A-6B are graphs showing tumor volume in mm³ over a period of 24 days post challenge (FIG. 6A) and survival over a period of 39 days (FIG. 6B) in groups of LTA-expressing and Empty Vector B16 tumors treated with LTA vaccine or EV at 15 μg on day 6, 15, and 24 as indicated by syringes in FIG. 6A.

FIG. 7 is a schematic diagram of enrichment of vaccine-specific T cell clonotypes derived from MCC patient samples.

FIGS. 8A-8H are graphs showing expression levels of CD14 (FIG. 8A), CD11b (FIG. 8B), CD11c (FIG. 8C), CD209 (FIG. 8D), CD80 (FIG. 8E), CD40 (FIG. 8F), HLA-DR (FIG. 8G), and CD1a (FIG. 8H) of monocyte-derived dendritic cells (Mo-DC) from MCC patient samples, incubated with either lipofectamine alone (Mo-DC Placebo) or lipofectamine with LTA mRNA vaccine.

FIGS. 9A-9C are graphs showing the number of T cells in culture (×10⁶) on day 28 following T cell stimulation with Placebo vs LTA vaccine Mo-DC (FIG. 9A), percent CD8⁺ cells out of total CD3⁺ cells (% CD8⁺ of CD3⁺) (FIG. 9B) and percent CD4⁺ cells out of total CD3⁺ cells (% CD4⁺ of CD3⁺) (FIG. 9C) over a period of 35 days following five pulses of T cell stimulation with Placebo vs LTA vaccine Mo-DC.

FIGS. 10A-10B are graphs showing percentage of PD-1⁺ cells and CD45RO⁺ cells out of CD8⁺ T cells on day 35 following T cell stimulation with Placebo vs LTA vaccine Mo-DC (FIG. 10A), and percentage of PD-1⁺ cells out of CD8⁺ T cells over a period of 35 days following T cell stimulation with Placebo vs LTA vaccine Mo-DC (FIG. 10B).

FIGS. 11A-11B are graphs showing IFN-γ release (pg/mL) from T cells stimulated with LTA vs Placebo Mo-DCs (FIG. 11A), and IFN-γ release (pg/mL) from LTA enriched T cells vs. Placebo T cells when exposed to matched tumor cells (FIG. 11B).

FIGS. 12A-12B are graphs showing tumor cell death as a fraction of total cells labeled with Cell Trace Violet using T cells expanded from Placebo vs LTA vaccine Mo-DC (FIG. 12A), and tumor cell killing relative to spontaneous release (Log 2 change) in a Europium-release killing assay in media control, spontaneous release, and T cells expanded from Placebo vs LTA vaccine Mo-DC (FIG. 12B).

FIGS. 13A-13C are graphs showing human in vitro vaccination killing response (FIG. 13A) and IFN-γ release assays (FIG. 13B-13C) using WAGA cell line exposed to LTA specific T cells and placebo T cells.

FIGS. 14A-14B are a schematic and a graph showing the expansion of LTA specific T cells compared to Placebo T cells after stimulating with LTA-vaccine-transfected DCs (LTA-Vax-DCs) and placebo-transfected DCs (Placebo-DCs). The clonotypes on the right side for FIG. 14B (SEQ ID NOS:27-46) represent the amino acid sequence of the TCR alpha and beta chain hypervariable regions corresponding to CDR3a and CDR3b separated by a dash.

FIGS. 15A-15D are images showing transcriptional states of T cells expanded with LTA vaccines and placebo. Distinct transcriptional states are enriched by LTA vaccine expansion including clusters 1 and 7 (FIG. 15A-15B). LTA vaccine-expanded clusters are enriched for cytotoxicity (FIG. 15C). Overnight restimulation also demonstrated preferential induction of proliferative states in the LTA-expanded T cells (FIG. 15D).

FIGS. 16A-16B are a pair of plots showing tumor growth and survival probability in B16-LTA murine model to evaluate synergy with current standard of care. Combination therapy with aPD1 resulted in increased growth suppression (FIG. 16A) and increased median survival (FIG. 16B) compared to LTA vaccine alone. N=10 mice per group.

FIGS. 17A-17E are plots showing the infiltration of immune cells following LTA vaccination in the dissected mice tumors compared to placeo tumors. As revealed by flow cytometry, significant increase is observed in total tumor immune infiltration (% CD45+ of live cells) (FIG. 17A), (% CD3+ of CD45+) (FIG. 17B), and cytotoxic potential (MFI GZMb in CD8+ and CD3+) (FIG. 17C) in LTA vaccine treated vs. placebo tumors. Additionally, significant increase was also observed in the proportion of dendritic cells (CD11c+ and MHCII+ of CD3− and CD19−) (FIG. 17D) and cDC1 marker XCR1 (FIG. 17E) in the LTA vaccine treated lymph nodes vs. placebo.

FIGS. 18A-18D are images showing LTA vaccine efficiency in single cloned B16-LTA tumor model. LTA expression was decreased among tumor cells that survived treatment with LTA mRNA vaccination compared to placebo (FIG. 18A). Single clone LTASC2 (FIG. 18B) showed increased growth suppression and increased survival (FIG. 18C-18D).

FIG. 19 is a plot showing complete tumor rejection in mice models after prophylactic vaccination using 15 μg of LTA mRNA vaccine compared to placebo group.

### DETAILED DESCRIPTION OF THE INVENTION

### I. Definitions

The terms “immunologic”, “immunological” or “immune” response refer to the development of a beneficial humoral (antibody mediated) and/or a cellular (mediated by antigen-specific T cells or their secretion products) response directed against an immunogen in a recipient patient. Such a response can be an active response induced by administration of immunogen or a passive response induced by administration of antibody or primed T-cells. The relative contributions of humoral and cellular responses to the protective or therapeutic effect of an immunogen can be distinguished by separately isolating antibodies and T-cells from an immunized syngeneic animal and measuring protective or therapeutic effect in a second subject.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered. The effect of the effective amount can be relative to a control. Such controls are known in the art and discussed herein, and can be, for example the condition of the subject prior to or in the absence of administration of the drug, or drug combination.

The terms “pharmaceutically acceptable” or “biocompatible” refer to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. The term “pharmaceutically acceptable salt” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; and N-benzylphenethylamine.

The term “biodegradable”, generally refers to a material that will degrade or erode under physiologic conditions to smaller units or chemical species that are capable of being metabolized, eliminated, or excreted by the subject. The degradation time is a function of composition and morphology of the material.

The terms “inhibit” or “reduce” generally mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%, or an integer there between. In some embodiments, the inhibition and reduction are compared at nucleic acid, protein, cell, tissue and/or organ levels.

The terms “treat” or “treatment” of a disease, disorder or condition refer to improving one or more symptoms or the general condition of a subject having the disease. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. In the case of an infectious disease, “treating” the infectious disease means reducing the load of the infectious agent in the subject.

The term “protect” or “protection of” a subject from developing a disease or from becoming susceptible to an infection means to partially or fully protect a subject from disease, infection and/or symptoms. As used herein to “fully protect” means that a treated subject does not develop a disease or infection caused by an agent such as Merkel cell polyomavirus. To “partially protect” as used herein means that a certain subset of subjects may be fully protected from developing a disease or infection after treatment, or that the subject does not develop a disease or infection with the same severity as an untreated subject.

The terms “prevent”, “prevention” or “preventing” mean to administer a composition or method to a subject or a system at risk for or having a predisposition for one or more symptom caused by a disease or disorder, to decrease the likelihood the subject will develop one or more symptoms of the disease or disorder, or to reduce the severity, duration, or time of onset of one or more symptoms of the disease or disorder.

The term “polynucleotide” or “nucleic acid” or “nucleic acid sequence” refers to a natural or synthetic molecule including two or more nucleotides linked by a phosphate group at the 3′ position of one nucleotide to the 5′ end of another nucleotide. The polynucleotide is not limited by length, and thus the polynucleotide can include deoxyribonucleic acid (DNA) or ribonucleic acid (RNA). Representative examples of the nucleic acids include bacterial plasmid vectors including expression, cloning, cosmid, and transformation vectors such as, but not limited to, viral vectors, vectors derived from bacteriophage nucleic acid, and synthetic oligonucleotides like chemically synthesized DNA or RNA. The term “nucleic acid” further includes modified or derivatized nucleotides and nucleosides such as, but not limited to, N-1-methylpseudouridine, halogenated nucleotides such as, but not only, 5-bromouracil, and derivatized nucleotides such as biotin-labeled nucleotides.

The term “gene” refers to a nucleic acid (e.g., DNA or RNA) sequence that comprises coding sequences necessary for the production of a polypeptide, RNA (e.g., including but not limited to, mRNA, tRNA and rRNA) or precursor. The polypeptide, RNA, or precursor can be encoded by a full-length coding sequence or by any portion thereof. The term also encompasses the coding region of a structural gene and the sequences located adjacent to the coding region on both the 5′ and 3′ ends for a distance of about 1 kb on either end such that the gene corresponds to the length of the full-length mRNA. The term “gene” encompasses both cDNA and genomic forms of a gene, which may be made of DNA, or RNA. A genomic form or clone of a gene may contain the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “nucleic acid molecule encoding,” refers to the order or sequence of nucleotides along a strand of nucleotides. The order of these nucleotides can determine the order of amino acids along the polypeptide (protein) chain. The nucleotide sequence can thus code for the amino acid sequence.

The term “expressed” or “expression” refers to the transcription from DNA to an RNA nucleic acid molecule at least complementary in part to a region of one of the two nucleic acid strands of the gene. The term “expressed” or “expression” also refers to the translation from said RNA nucleic acid molecule to give a protein or polypeptide or a portion thereof.

The term “antigen” refers to any substance (e.g., peptide, protein, nuclei acid, lipid, small molecule, such as a moiety expressed by or otherwise associated with a pathogen or cancerous or pre-cancerous cell) that serves as a target for the receptors of an adaptive immune response. The antigen may be a structural component of a pathogen, cancerous or pre-cancerous cell.

The term “polypeptide” refers to a chain of amino acids of any length, regardless of modification (e.g., phosphorylation or glycosylation). In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

As used herein, a “variant,” “mutant,” or “mutated” polynucleotide contains at least one polynucleotide sequence alteration as compared to the polynucleotide sequence of the corresponding wild-type or parent polynucleotide. Mutations may be natural, deliberate, or accidental. Mutations include substitutions, deletions, and insertions.

As used herein, an “adjuvant” is a substance that increases the ability of an antigen to stimulate the immune system.

As used herein, the term “carrier” or “excipient” refers to an organic or inorganic ingredient, natural or synthetic inactive ingredient in a formulation, with which one or more active ingredients are combined.

The terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx.+/−10%; in other forms the values may range in value either above or below the stated value in a range of approx.+/−5%; in other forms the values may range in value either above or below the stated value in a range of approx.+/−2%; in other forms the values may range in value either above or below the stated value in a range of approx.+/−1%. The preceding ranges are intended to be made clear by context, and no further limitation is implied.

Every compound disclosed herein is intended to be and should be considered to be specifically disclosed herein. Further, every subgroup that can be identified within this disclosure is intended to be and should be considered to be specifically disclosed herein. As a result, it is specifically contemplated that any compound, or subgroup of compounds can be either specifically included for or excluded from use or included in or excluded from a list of compounds.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular polypeptide is disclosed and discussed and a number of modifications that can be made to a number of polypeptides are discussed, specifically contemplated is each and every combination and permutation of polypeptides and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### II. Compositions

Compositions for eliciting an immune response in a subject to confer resistance to subsequent exposure to infectious agents, or to enhance an immune response a pre-existing antigen, such as a tumor antigen in a subject with cancer are described.

In some embodiments, compositions include one or more viral antigens derived from one or more viruses that cause cancer. In other embodiments, compositions include one or more immunogenic domains and fragments of viral antigens derived from one or more viruses that cause cancer. As used in this context, a viral antigen is derived from a virus if its sequence originates from the virus. For example, the antigen can be a fragment of, or a full length, viral protein. The antigen can also be modified relative to the fragment or full-length protein, for example amino acid addition(s), deletion(s), or substitution(s). In some embodiments, the antigen has at least 50%, 60%, 70%, 75%, 80%, 85%, 90%, or 95% sequence identity to the fragment or full-length viral protein from which is derived.

Exemplary viruses that cause cancer include Merkel Cell Polyomavirus (MCPyV), Hepatitis B virus (HBV), Hepatitis C virus (HCV), Human papilloma virus (HPV), Kaposi's sarcoma-associated herpesvirus, Epstein-Barr virus (EBV), and human T-cell lymphotropic virus.

Tumor vaccines for MCC and other cancers have largely been ineffective. This is likely due to i) challenges in predicting antigens that will drive effective T cell immunity and ii) generating high levels of T cell expansion of effector and memory populations. Thus, in preferred embodiments, the compositions include one or more viral antigens that drive effective T cell anti-tumor immunity and/or generate high levels of T cell expansion of effector and memory populations.

In preferred embodiments, compositions include one or more nucleic acids encoding viral antigens derived from one or more viruses that cause cancer, optionally formulated with carriers such as liposomes, and polymeric micro- and nanoparticles.

### A. Viral Antigens

Antigens for use in the disclosed compositions and methods are provided. The antigen can be or can include, for example, peptides, proteins, polysaccharides, saccharides, lipids, nucleic acids, small molecules (alone or with a hapten), or combinations thereof. In preferred embodiments, the antigen is a polypeptide, preferably a polypeptide derived from the virus against which the immune response is desired.

### 1. Merkel Cell Polyomavirus

It has been established that one or more viral antigens of Merkel Cell Polyomavirus (MCPyV), in particular LTA or STA, or an immunogenic fragment thereof, is effective to elicit immune responses against MCPyV-driven Merkel cell carcinoma. Thus, immunogenic compositions of one or more viral antigens of MCPyV for eliciting immune responses against MCPyV-driven Merkel cell carcinoma are described.

MCPyV is a non-enveloped, double-stranded DNA virus that is a part of the normal microbiome for a majority of individuals in endemic areas (Kervarrec T, et al., Front Oncol. 2019 Jun. 10; 9:451). Recent investigation has shown that a coding region for a truncated form of the viral Large T Antigen (LTA) and its isoform the Small T Antigen (STA) are integrated into the genome of MCPyV associated MCC and causes malignant transformation through mechanisms including an interaction between LTA and the Retinoblastoma Protein (RB1). Furthermore, expression of the T antigens in MCPyV associated MCC is required to maintain proliferation of the cancer (Hesbacher S et al., Oncotarget. 2016 May 31; 7(22):32956-68).

In preferred embodiments, the viral antigen is viral LTA derived from MCPyV.

The viral antigen preferably is a nucleic acid (e.g., mRNA) encoding one or more polypeptides of MCPyV.

In preferred embodiments, the viral antigen derived from LTA of MCPyV includes the amino acid sequence of SEQ ID NO:1.

MDLVLNRKEREALCKLLEIAPNCYGNIPLMKAAFKRSCLKHHPDKGGNPVI MMELNTLWSKFQQNIHKLRSDFSMFDEVDEAPIYGTTKFKEWWRSGGFSFG KAYEYGPNPHGTNSRSRKPSSNASRGAPSGSSPPHSQSSSSGYGSFSASQA SDSQSRGPDIPPEHHEEPTSSSGSSSREETTNSGRESSTPNGTSVPRNSSR TDGTWEDLFCDESLSSPEPPSSSEEPEEPPSSRSSPRQPPSSSAEEASSSQ FTD (SEQ ID NO: 1), or a fragment or variant thereof with e.g., at least 50%, 60%, 70%, 75%, 80%, 85%, 90%, or 95% sequence identity thereto.

In some embodiments, the viral antigen is STA derived from MCPyV, or an antigenic fragment thereof. In preferred embodiments, the viral antigen derived from STA of MCPyV includes the amino acid sequence of SEQ ID NO:2.

MDLVLNRKEREALCKLLEIAPNCYGNIPLMKAAFKRSCLKHHPDKGGNPVI MMELNTLWSKFQQNIHKLRSDFSMFDEVSTKFPWEEYGTLKDYMQSGYNAR FCRGPGCMLKQLRDSKCACISCKLSRQHCSLKTLKQKNCLTWGECFCYQCF ILWFGFPPTWESFDWWQKTLEETDYCLLHLHLF (SEQ ID NO:2), or a fragment or variant thereof with e.g., at least 50%, 60%, 70%, 75%, 80%, 85%, 90%, or 95% sequence identity thereto.

### 2. Antigens for Other Viruses

Alternatively, antigen can be designed for use against Hepatitis B virus (HBV), Hepatitis C virus (HCV), Human papilloma virus (HPV), Kaposi's sarcoma-associated herpesvirus, Epstein-Barr virus (EBV), or human T-cell lymphotropic virus. In preferred embodiments, the antigen is a polypeptide derived from the target virus. Exemplary antigens include, but are not limited to, HPV E2, E5, E6, and antigenic fragments thereof.

In one embodiment, the viral antigen is HPV E2, or an antigenic fragment thereof. In preferred embodiments, the viral antigen derived from HPV E2 protein includes the amino acid sequence of SEQ ID NO:3 as follows.

METLCQRLNVCQDKILTHYENDSTDLRDHIDYWKHMRLECAIYYKAREMGF KHINHQVVPTLAVSKNKALQAIELQLTLETIYNSQYSNEKWTLQDVSLEVY LTAPTGCIKKHGYTVEVQFDGDICNTMHYTNWTHIYICEEASVTVVEGQVD YYGLYYVHEGIRTYFVQFKDDAEKYSKNKVWEVHAGGQVILCPTSVFSSNE VSSPEIIRQHLANHPAATHTKAVALGTEETQTTIQRPRSEPDTGNPCHTTK LLHRDSVDSAPILTAFNSSHKGRINCNSNTTPIVHLKGDANTLKCLRYRFK KHCTLYTAVSSTWHWTGHNVKHKSAIVTLTYDSEWQRDQFLSQVKIPKTIT VSTGFMSI (SEQ ID NO:3), or a fragment or variant thereof with e.g., at least 50%, 60%, 70%, 75%, 80%, 85%, 90%, or 95% sequence identity thereto.

In one embodiment, the viral antigen is HPV E5, or an antigenic fragment thereof. In preferred embodiments, the viral antigen derived from HPV E5 protein includes the amino acid sequence of SEQ ID NO:4 as follows.

MTNLDTASTTLLACFLLCFCVLLCVCLLIRPLLLSVSTYTSLILLVXVLWI TAASAFRCF IVYIVFVYIPLFLIHTHARFLIT (SEQ ID NO:4), or a fragment or variant thereof with e.g., at least 50%, 60%, 70%, 75%, 80%, 85%, 90%, or 95% sequence identity thereto.

In one embodiment, the viral antigen is HPV E6, or an antigenic fragment thereof. In preferred embodiments, the viral antigen derived from HPV E6 protein includes the amino acid sequence of SEQ ID NO:5 as follows.

MHQKRTAMFQDPQERPRKLPQLCTELQTTIHDIILECVYCKQQLLRREVYD FAFRDLCIVYRDGNPYAVCDKCLKFYSKISEYRHYCYSLYGTTLEQQYNKP LCDLLIRCINCQKPLCPEEKQRHLDKKQRFHNIRGRWTGRCMSCCRSSRTR RETQL (SEQ ID NO:5), or a fragment or variant thereof with e.g., at least 50%, 60%, 70%, 75%, 80%, 85%, 90%, or 95% sequence identity thereto.

All the polypeptide sequences disclosed herein are expressly provided both with and without the N-terminal methionine (M). Thus, in some embodiments, the antigen is or includes the amino acid sequence of any one of SEQ ID NOS:1-5, without the N-terminal methionine.

### B. Isolated Nucleic Acids

### 1. Nucleic Acids Encoding Antigens

Isolated nucleic acid sequences encoding antigens, including, but not limited to, the LTA protein and/or immunogenic domains and fragments thereof and other antigens disclosed herein, and vectors and other expression constructs encoding the foregoing are also disclosed.

In some embodiments, the isolated nucleic acids include nucleotide sequence encoding one or more viral proteins of MCPyV, Hepatitis B virus (HBV), Hepatitis C virus (HCV), Human papilloma virus (HPV), Kaposi's sarcoma-associated herpesvirus, Epstein-Barr virus (EBV), or human T-cell lymphotropic virus.

In preferred embodiments, the isolated nucleic acids include nucleotide sequence encoding the LTA protein of MCPyV, and/or immunogenic domains and fragments or variants thereof.

In some embodiments, the isolated nucleic acid encoding the LTA protein of MCPyV has the following nucleotide sequence.

(SEQ ID NO: 6)
ATGGATTTAGTCCTAAATAGGAAAGAAAGAGAGGCTCTCTGCAAGCTTTTA
GAGATTGCTCCTAATTGTTATGGCAACATCCCTCTGATGAAAGCTGCTTTC
AAAAGAAGCTGCTTAAAGCATCACCCTGATAAAGGGGGAAATCCTGTTATA
ATGATGGAATTGAACACCCTTTGGAGCAAATTCCAGCAAAATATCCACAAG
CTCAGAAGTGACTTCTCTATGTTTGATGAGGTCGACGAGGCCCCTATATAT
GGGACCACTAAATTCAAAGAATGGTGGAGATCAGGAGGATTCAGCTTCGGG
AAGGCATACGAATATGGGCCCAATCCACACGGGACCAACTCAAGATCCAGA
AAGCCTTCCTCCAATGCATCCAGGGGAGCCCCCAGTGGAAGCTCACCACCC
CACAGCCAGAGCTCTTCCTCTGGGTATGGGTCCTTCTCAGCGTCCCAGGCT
TCAGACTCCCAGTCCAGAGGACCCGATATACCTCCCGAACACCATGAGGAA
CCCACCTCATCCTCTGGATCCAGTAGCAGAGAGGAGACCACCAATTCAGGA
AGAGAATCCAGCACACCCAATGGAACCAGTGTACCTAGAAATTCTTCCAGA
ACGGATGGCACCTGGGAGGATCTCTTCTGCGATGAATCACTTTCCTCCCCT
GAGCCTCCCTCGTCCTCTGAGGAGCCTGAGGAGCCCCCCTCCTCAAGAAGC
TCGCCCCGGCAGCCCCCGTCTTCCTCTGCCGAGGAGGCCTCGTCATCTCAG
TTTACAGATTAG

The nucleotide sequence of SEQ ID NO:6 encodes a polypeptide represented by SEQ ID NO:1. In some embodiments, the isolated nucleic acid includes nucleotide sequence (e.g., mRNA) that encodes a polypeptide represented by SEQ ID NO:1, or a variant or fragment thereof having more than 50%, 60%, 70%, 75%, 80%, 85%, 90%, or 95% sequence identity to the amino acid sequence of SEQ ID NO:1, such as, but not limited to, SEQ ID NO:6.

In other embodiments, the isolated nucleic acids include a nucleotide sequence encoding the STA protein of MCPyV, and/or immunogenic domains and fragments or variants thereof. In some embodiments, the isolated nucleic acid encoding the STA protein of MCPyV has the following nucleotide sequence.

(SEQ ID NO: 7)
ATGGATTTAGTCCTAAATAGGAAAGAAAGAGAGGCTCTCTGCAAGCTTTTA
GAGATTGCTCCTAATTGTTATGGCAACATCCCTCTGATGAAAGCTGCTTTC
AAAAGAAGCTGCTTAAAGCATCACCCTGATAAAGGGGGAAATCCTGTTATA
ATGATGGAATTGAACACCCTTTGGAGCAAATTCCAGCAAAATATCCACAAG
CTCAGAAGTGACTTCTCTATGTTTGATGAGGTCAGTACAAAATTTCCTTGG
GAAGAATATGGAACTTTAAAGGATTATATGCAAAGTGGATATAATGCTAGA
TTTTGCAGAGGTCCTGGGTGCATGCTTAAGCAACTTAGAGATTCTAAGTGC
GCTTGTATTAGCTGTAAGTIGTCTCGCCAGCATTGTAGTCTAAAAACTTTA
AAGCAAAAAAACTGTCTGACGTGGGGAGAGTGTTTTTGCTATCAGTGCTTT
ATTCTTTGGTTTGGATTTCCTCCTACTTGGGAAAGTTTTGACTGGTGGCAA
AAAACTTTAGAAGAAACTGACTACTGCTTACTGCATCTGCACCTTTTCTA
G.

The nucleotide sequence of SEQ ID NO:7 encodes a polypeptide represented by SEQ ID NO:2. In some embodiments, the isolated nucleic acid includes nucleotide sequence (e.g., mRNA) that encodes a polypeptide represented by SEQ ID NO:2, or a variant or fragment thereof having more than 50%, 60%, 70%, 75%, 80%, 85%, 90%, or 95% sequence identity to the amino acid sequence of SEQ ID NO:2, such as, but not limited to, SEQ ID NO:7.

In some embodiments, the isolated nucleic acids include nucleotide sequence encoding the E2 protein of HPV, and/or immunogenic domains and fragments or variants thereof. In some embodiments, the isolated nucleic acid encoding the HPV E2 protein is as follows.

(SEQ ID NO: 8)
ATGGAGACTCTTTGCCAACGTTTAAATGTGTGTCAGGACAAAATACTAACA
CATTATGAAAATGATAGTACAGACCTACGTGACCATATAGACTATTGGAAA
CACATGCGCCTAGAATGTGCTATTTATTACAAGGCCAGAGAAATGGGATTT
AAACATATTAACCACCAGGTGGTGCCAACACTGGCTGTATCAAAGAATAAA
GCATTACAAGCAATTGAACTGCAACTAACGTTAGAAACAATATATAACTCA
CAATATAGTAATGAAAAGTGGACATTACAAGACGTTAGCCTTGAAGTGTAT
TTAACTGCACCAACAGGATGTATAAAAAAACATGGATATACAGTGGAAGTG
CAGTTTGATGGAGACATATGCAATACAATGCATTATACAAACTGGACACAT
ATATATATTTGTGAAGAAGCATCAGTAACTGTGGTAGAGGGTCAAGTTGAC
TATTATGGTTTATATTATGTTCATGAAGGAATACGAACATATTTTGTGCAG
TTTAAAGATGATGCAGAAAAATATAGTAAAAATAAAGTATGGGAAGTTCAT
GCGGGTGGTCAGGTAATATTATGTCCTACATCTGTGTTTAGCAGCAACGAA
GTATCCTCTCCTGAAATTATTAGGCAGCACTTGGCCAACCACCCCGCCGCG
ACCCATACCAAAGCCGTCGCCTTGGGCACCGAAGAAACACAGACGACTATC
CAGCGACCAAGATCAGAGCCAGACACCGGAAACCCCTGCCACACCACTAAG
TTGTTGCACAGAGACTCAGTGGACAGTGCTCCAATCCTCACTGCATTTAAC
AGCTCACACAAAGGACGGATTAACTGTAATAGTAACACTACACCCATAGTA
CATTTAAAAGGTGATGCTAATACTTTAAAATGTTTAAGATATAGATTTAAA
AAGCATTGTACATTGTATACTGCAGTGTCGTCTACATGGCATTGGACAGGA
CATAATGTAAAACATAAAAGTGCAATTGTTACACTTACATATGATAGTGAA
TGGCAACGTGACCAATTTTTGTCTCAAGTTAAAATACCAAAAACTATTACA
GTGTCTACTGGATTTATGTCTATATGA.

The nucleotide sequence of SEQ ID NO:8 encodes a polypeptide represented by SEQ ID NO:3. In some embodiments, the isolated nucleic acid includes nucleotide sequence (e.g., mRNA) that encodes a polypeptide represented by SEQ ID NO:3, or a variant or fragment thereof having more than 50%, 60%, 70%, 75%, 80%, 85%, 90%, or 95% sequence identity to the amino acid sequence of SEQ ID NO:3, such as, but not limited to, SEQ ID NO:8.

In one embodiment, the viral antigen is HPV E5, or an antigenic fragment thereof. Exemplary nucleic acid sequence encoding HPV E5 protein is as follows.

(SEQ ID NO: 9)
ATGACAAATCTTGATACTGCATCCACAACATTACTGGCGTGCTTTTTGCTT
TGCTTTTGTGTGCTTTTGTGTGTCTGCCTATTAATACGTCCGCTGCTTTTG
TCTGTGTCTACATACACATCATTATTTTTAATACATACACATGCACGCTTT
TTAATTACATAA.

The nucleotide sequence of SEQ ID NO:9 encodes a polypeptide represented by SEQ ID NO:4. In some embodiments, the isolated nucleic acid includes nucleotide sequence (e.g., mRNA) that encodes a polypeptide represented by SEQ ID NO:4, or a variant or fragment thereof having more than 50%, 60%, 70%, 75%, 80%, 85%, 90%, or 95% sequence identity to the amino acid sequence of SEQ ID NO:4, such as, but not limited to, SEQ ID NO:9.

In one embodiment, the viral antigen is HPV E6, or an antigenic fragment thereof. Exemplary nucleic acid sequence encoding HPV E6 protein is as follows.

(SEQ ID NO: 10)
ATGCACCAAAAGAGAACTGCAATGTTTCAGGACCCACAGGAGCGACCCAGA
AAGTTACCACAGTTATGCACAGAGCTGCAAACAACTATACATGATATAATA
TTAGAATGTGTGTACTGCAAGCAACAGITACTGCGACGTGAGGTATATGAC
TTTGCTTTTCGGGATTTATGCATAGTATATAGAGATGGGAATCCATATGCT
GTATGTGATAAATGTTTAAAGTTTTATTCTAAAATTAGTGAGTATAGACAT
TATTGTTATAGTTTGTATGGAACAACATTAGAACAGCAATACAACAAACCG
TTGTGTGATTTGTTAATTAGGTGTATTAACTGTCAAAAGCCACTGTGTCCT
GAAGAAAAGCAAAGACATCTGGACAAAAAGCAAAGATTCCATAATATAAGG
GGTCGGTGGACCGGTCGATGTATGTCTTGTTGCAGATCATCAAGAACACGT
AGAGAAACCCAGCTGTAA.

The nucleotide sequence of SEQ ID NO:10 encodes a polypeptide represented by SEQ ID NO:5. In some embodiments, the isolated nucleic acid includes nucleotide sequence (e.g., mRNA) that encodes a polypeptide represented by SEQ ID NO:5, or a variant or fragment thereof having more than 50%, 60%, 70%, 75%, 80%, 85%, 90%, or 95% sequence identity to the amino acid sequence of SEQ ID NO:5, such as, but not limited to, SEQ ID NO:10.

In some embodiments, the isolated nucleic acid sequence includes nucleotide sequence encoding antigens, such as, but not limited to the LTA or STA protein of MCPyV, and/or immunogenic domains and fragments thereof, and optionally one or more additional elements linked thereto. The elements include but are not limited to elements that enhance transcription and/or translation and/or purification of the antigen.

Non-limiting exemplary elements are discussed below and include, but are not limited to, (i) signal peptide, (ii) one or more restriction sites, (iii) promoter region such as T7 promoter, (iv) TRILINK CAP site, (v) traditional KOZAK sequence, (vi) 5′ untranslated region (UTR), (vii) 3′ UTR, and (viii) poly(A) tail.

It will be appreciated that some of these elements are present only for the purpose of transcription (e.g., promoter, CAP site, UTRs, etc.), and thus, may be absent from an mRNA construct and expressed protein. Furthermore, it will also be appreciated that some of these elements are present only for nucleic acid stability and/or translation (e.g., TRILINK CAP site, KOZAK sequence, poly(A) tail) and thus may be absent from the expressed protein. Furthermore, it will be appreciated that some of the elements are present only for protein trafficking and/or purification (e.g., signal sequence and purification tags), and may be present or absent in the final antigenic polypeptide. Thus, these elements and others are disclosed in all combinations, and can be selectively present or absent from the disclosed constructs depending on the particular composition at a particular time, e.g., DNA or RNA vector construct, mRNA, or protein.

### 2. Signal Peptide

In some embodiments, a signal peptide is incorporated to improve protein expression of one or more viral antigens encoded in the nucleic acid construct. Thus, in some embodiments, the nucleic acid sequence also includes one or more signal peptides.

The spike protein of the coronavirus requires a signal peptide to guide its transportation to its membrane destination. The signal peptide has the first 13 amino acids with helix-forming high-hydrophobicity residues. In some embodiments, the signal peptide sequence is derived from the spike protein of a coronavirus variant of SARS-CoV-2, such as SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant). In some embodiments, any leading N-terminal sequence including a positively charged “N” region, a hydrophobic helical leucine rich “H” region, and an uncharged alanine rich “C” region with a cleavage site is used.

An exemplary nucleotide sequence encoding a signal peptide sequence from the SARS-CoV-2 spike protein is represented by SEQ ID NO:11.

(SEQ ID NO: 11)
ATGTTCGTGTTCCTGGTGCTGCTGCCCCTGGTGAGCAGCCAGTGCGTG.

The nucleotide sequence of SEQ ID NO:11 encodes a polypeptide having amino acid sequence of MFVFLVLLPLVSSQCV (SEQ ID NO:12).

An exemplary embodiment is a nucleic acid encoding a polypeptide including a signal peptide sequence from the SARS-CoV-2 spike protein leading into the nucleotide sequence encoding the antigens, such as, but not limited to, LTA protein of MCPyV, and/or immunogenic domains and fragments thereof.

### 3. Promoter Sequence

In some embodiments, the nucleic acid includes a promoter sequence. In one embodiment, the nucleic acid includes a T7 promoter sequence such as TAATACGACTCACTATAAG (SEQ ID NO:13). In another embodiment, the nucleic acid includes a T3 promoter having the nucleotide sequence of ATTAACCCTCACTAAAG (SEQ ID NO:14). In a further embodiment, the nucleic acid includes a SP6 promoter having the nucleotide sequence of

(SEQ ID NO: 15)
ATTTAGGTGACACTATAG.

### 4. 5′ UTR

In some embodiments, the nucleic acid includes a 5′ untranslated region (UTR) sequence.

As shown in the Examples, for the 5′ UTR, a synthetic sequence was chosen based on an earlier study (Cao, J et al., Nat Commun 12, 4138 (2021)). This sequence was generated by artificial intelligence to be improved for high efficiency translation and was among the highest performers in their 5′ UTR screen.

In one embodiment, the nucleic acid includes a 5′ UTR sequence as represented by SEQ ID NO:16.

(SEQ ID NO: 16)
ACCCAAGCTGGCTAGCGTTTAAACTTAAGCTTGGTACCGCTTGTCTCGCTC
CGGGGAACGCTCGGAAACTCCCGGCCGCCGCCACCCGCGTCTGTTCTGTTA
CACAAGGGAAGAAAAGCCGCTGCCGCACTCCGAGTGT.

In some embodiments, the nucleic acid includes a 5′ UTR sequence selected from the following.

B2M 5′ UTR:
(SEQ ID NO: 17)
CAACAACAACAACAACAACAACAACAACAACAACAACAACAACAACAACAA
CAACAACAACAACAAGGGCATTCCTGAAGCTGACAGCATTCGGGCCGAGGC
CACC
MB 5′ UTR
(SEQ ID NO: 18)
AGACCAGTTCTTAGCCATCAAGCAGAGACTCTGAAGCCAGACTACCTGGGT
CCCAATCTTGGGCTTGGTATTTCCTCGCTGTGTGACTCTGGACTGCGCCGC
CACC
MCPyV 5′ UTR:
(SEQ ID NO: 19)
GGGGCTCCTAGCCTCCGAGGCCTCTGGAAAAAAAAGAGAGAGGCCTCTGAG
GCTTAAGAGGCTTAATTAGCAAAAAAGGCAGTATCTAAGGGCAGATCCCAA
GGGCGGGAAACTGCAGTATAAAAACCACTCCTTAGTGAGGTAGCTCATTTG
CTCCTCTGCTCTTTCTGCAAACTCCTTCTGCATATAGACAAG

### 5. 3′ UTR

In some embodiments, the nucleic acid includes a 3′ UTR sequence.

As shown in the Examples, the natural 3′ UTR from the Human HBA1 gene was selected because of its demonstrated success in prior mRNA constructs and short length, which is predicted to minimize secondary structure formation and facilitate translation while preventing or delaying degradation.

In one embodiment, the nucleic acid includes a 3′ UTR sequence as represented by SEQ ID NO:20.

(SEQ ID NO: 20)
GCTGGAGCCTCGGTGGCCTAGCTTCTTGCCCCTTGGGCCTCCCCCCAGCCC
CTCCTCCCCTTCCTGCACCCGTACCCCCGTGGTCTTTGAATAAAGTCTGAG
TGGGCGGCA.

### 6. Affinity Tag

In some embodiments, the isolated nucleic acids also include affinity tags facilitate purification and rapid detection. Exemplary affinity tags include a FLAG-tag e.g., having the amino acid sequence DYKDDDDK (SEQ ID NO:21), His-tag having e.g., 6 or more histidine residues, haemagglutinin (HA) tag, MYC tag, a Spot-tag having, e.g., the amino acid sequence PDRVRAVSHWSS (SEQ ID NO:22), C-tag having the amino acid sequence EPEA, and Strep-Tag having the amino acid sequence WSHPQFEK (SEQ ID NO:23).

In some embodiments, the isolated nucleic acid sequence includes nucleotide sequence encoding the antigen such as the LTA protein of MCPyV, and/or immunogenic domains and fragments thereof, and (i) signal peptide at the 5′ end of the nucleotide sequence encoding the antigen, (ii) one or more restriction sites flanking either or both side of the nucleotide sequence, (iii) promoter region such as T7 promoter, (iv) TRILINK CAP site, (v) a KOZAK sequence, (vi) 5′ UTR, (vii) 3′ UTR, and (viii) poly(A) tail.

In one embodiment, the isolated nucleic acid sequence includes nucleotide sequence represented by SEQ ID NO:24.

(SEQ ID NO: 24)
GAGCTCACGCGTTAATACGACTCACTATAAGACCCAAGCTGGCTAGCGTTT
AAACTTAAGCTTGGTACCGCTTGTCTCGCTCCGGGGAACGCTCGGAAACTC
CCGGCCGCCGCCACCCGCGTCTGTTCTGTTACACAAGGGAAGAAAAGCCGC
TGCCGCACTCCGAGTGTGCCACCAtgTTCGTGTTCCTGGTGCTGCTGCCCC
TGGTGAGCAGCCAGTGCGTGgatttagtcctaaataggaaagaaagagagg
ctctctgcaagcttttagagattgctcctaattgttatggcaacatccctc
tgatgaaagctgctttcaaaagaagctgcttaaagcatcaccctgataaag
ggggaaatcctgttataatgatggaattgaacaccctttggagcaaattcc
agcaaaatatccacaagctcagaagtgacttctctatgtttgatgaggtcg
acgaggcccctatatatgggaccactaaattcaaagaatggtggagatcag
gaggattcagcttcgggaaggcatacgaatatgggcccaatccacacggga
ccaactcaagatccagaaagccttcctccaatgcatccaggggagccccca
gtggaagctcaccaccccacagccagagctcttcctctgggtatgggtcct
tctcagcgtcccaggcttcagactcccagtccagaggacccgatatacctc
ccgaacaccatgaggaacccacctcatcctctggatccagtagcagagagg
agaccaccaattcaggaagagaatccagcacacccaatggaaccagtgtac
ctagaaattcttccagaacggatggcacctgggaggatctcttctgcgatg
aatcactttcctcccctgagcctccctcgtectctgaggagcctgaggagc
ccccctcctcaagaagctcgccccggcagcccccgtcttcctctgccgagg
aggcctcgtcatctcagtttacagattagGCTGGAGCCTCGGTGGCCTAGC
TTCTTGCCCCTTGGGCCTCCCCCCAGCCCCTCCTCCCCTTCCTGCACCCGT
ACCCCCGTGGTCTTTGAATAAAGTCTGAGTGGGCGGCAAAAAAAAAAAAAA
AAAAAAAAAAAAAAAAAAAAAAAAAAAAAAAAAAAAAAAAAAAAAAAAAAA
AAAAAAAAAAAAAAAAAAAAAAAAAAAAAAAAAAAATCTAGAGAGCTC.

The nucleotide sequence of SEQ ID NO:24 includes an open reading frame (bolded) that encodes a polypeptide represented by SEQ ID NO:25 which includes a signal peptide sequence (italics).

(SEQ ID NO: 25)
MFVFLVLLPLVSSQCVDLVLNRKEREALCKLLEIAPNCYGNIPLMKAAFKR
SCLKHHPDKGGNPVIMMELNTLWSKFQQNIHKLRSDFSMFDEVDEAPIYGT
TKFKEWWRSGGFSFGKAYEYGPNPHGINSRSRKPSSNASRGAPSGSSPPHS
QSSSSGYGSFSASQASDSQSRGPDIPPEHHEEPTSSSGSSSREETTNSGRE
SSTPNGTSVPRNSSRTDGTWEDLFCDESLSSPEPPSSSEEPEEPPSSRSSP
RQPPSSSAEEASSSQFTD.

As used herein, “isolated nucleic acid” refers to a nucleic acid that is separated from other nucleic acid molecules that are present in a mammalian genome, including nucleic acids that normally flank one or both sides of the nucleic acid in a mammalian genome. The term “isolated” as used herein with respect to nucleic acids also includes the combination with any non-naturally occurring nucleic acid sequence, since such non-naturally occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

The nucleic acid sequences encoding the disclosed proteins and polypeptides can be or include, for example, engineered genomic sequences and fragments of naturally occurring genomic sequence, mRNA sequence wherein the exons have been deleted, and other nucleic acid sequences. Nucleic acids encoding the viral proteins (e.g., antigens including, but not limited to, LTA proteins) and domains thereof may be optimized for expression in the expression host of choice. Codons may be substituted with alternative codons encoding the same amino acid to account for differences in codon usage different host organisms. In this manner, the nucleic acids may be synthesized using expression host-preferred codons.

Nucleic acids can be in sense or antisense orientation, or can be complementary to a reference sequence, e.g., encoding the antigen such as LTA protein or immunogenic domain(s) thereof. Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Common modifications are discussed in more detail below.

Nucleic acids encoding polypeptides can be administered to subjects in need thereof. Nucleic delivery involves introduction of “foreign” nucleic acids into a cell and ultimately, into a live animal. Compositions and methods for delivering nucleic acids to a subject are known in the art (see Understanding Gene Therapy, Lemoine, N. R., ed., BIOS Scientific Publishers, Oxford, 2008).

Nucleic acids may also be delivered by other carriers, including liposomes, and polymeric micro- and nanoparticles.

### C. Nanoparticles

In some embodiments, compositions of one or more viral antigens, including, but not limited to, Merkel Cell Polyomavirus antigens, for eliciting immune responses against, e.g., virally derived cancers such as Merkel cell polyomavirus-driven Merkel cell carcinoma include one or more particles for delivery into the body.

Appropriate delivery vehicles for the compounds are known in the art and can be selected to suit the particular antigen compositions. For example, in some embodiments, the composition is incorporated into or encapsulated by, or bound to, a nanoparticle, microparticle, microsphere, micelle, natural or synthetic lipoprotein particle, liposomal nanoparticle, or dendrimeric particle. In other embodiments, the composition is incorporated into or encapsulated by or bound to one or more cationic polymers.

In preferred embodiments, the composition is incorporated into lipid nanoparticles. In one embodiment, the composition is incorporated into lipid nanoparticles formed with commercially available SM-102, 1,2-DSPC, cholesterol, and DMG-PEG, preferably in a lipid molar ratio of 50:10:38.5:1.5.

### 1. Lipidic Particles

In some embodiments, the particle is a lipid particle, liposome, or micelle, or includes a lipid core. Lipid particles and lipid nanoparticles are known in the art. Lipid particles are formed from one or more lipids, which can be neutral, anionic, or cationic at physiologic pH. The lipid particle is preferably made from one or more biocompatible lipids. The lipid particles may be formed from a combination of more than one lipid, for example, a charged lipid may be combined with a lipid that is non-ionic or uncharged at physiological pH.

Representative neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol.

Representative cationic lipids include, but are not limited to, N-[1-(2,3-dioleoyloxy)propyl]-N,N,N-trimethyl ammonium salts, referred to as TAP lipids, for example, methylsulfate salt. Representative TAP lipids include, but are not limited to, DOTAP (dioleoyl-), DMTAP (dimyristoyl-), DPTAP (dipalmitoyl-), and DSTAP (distearoyl-). Representative cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N—(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

a. Micelles

In some embodiments, the particle or particle core is a lipid micelle. Lipid micelles can be formed, for instance, as a water-in-oil emulsion with a lipid surfactant. An emulsion is a blend of two immiscible phases wherein a surfactant is added to stabilize the dispersed droplets. In some embodiments the lipid micelle is a microemulsion. A microemulsion is a thermodynamically stable system composed of at least water, oil and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents.

b. Liposomes

In some embodiments, the particle or particle core is a liposome. Liposomes are small vesicles composed of an aqueous medium surrounded by lipids arranged in spherical bilayers. Liposomes can be classified as small unilamellar vesicles, large unilamellar vesicles, or multi-lamellar vesicles. Multi-lamellar liposomes contain multiple concentric lipid bilayers. Liposomes can be used to encapsulate targeted agents, by trapping hydrophilic agents in the aqueous interior or between bilayers, or by trapping hydrophobic agents within the bilayer.

The lipid micelles and liposomes typically have an aqueous center. The aqueous center can contain water or a mixture of water and alcohol. Representative alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

In one embodiment, liposomes are prepared from long chain fatty acids and phytosterol formulations.

c. Solid Lipid Particles

In some embodiments, the particle is a solid lipid particle, or includes a solid lipid core. Solid lipid particles present an alternative to the colloidal micelles and liposomes. Solid lipid particles are typically submicron in size, i.e., from about 10 nm to about 1 micron, from 10 nm to about 500 nm, or from 10 nm to about 250 nm. Solid lipid particles are formed of lipids that are solids at room temperature. They are derived from oil-in-water emulsions, by replacing the liquid oil by a solid lipid.

Representative solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, preferably 12-30 carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Representative solid lipids can include cetyl palmitate or beeswax. Cyclodextrin can also be used.

### D. Adjuvants

The disclosed compositions can include one or more adjuvants. Suitable adjuvants are known in the art.

Exemplary adjuvants include, but are not limited to, aluminum hydroxide, aluminum phosphate, emulsion adjuvants, MF59, and AS03. LR agonists have been extensively studied as vaccine adjuvants. CpG, Poly I:C, glucopyranosyl lipid A (GLA), and resiquimod (R848) are agonists for TLR9, TLR3, TLR4, and TLR7/8, respectively. In some embodiments, adjuvants are one or more of STING agonists, RIG-I agonist, and montanide.

In some embodiments, double-stranded RNAs that are produced as a byproduct of the in vitro transcription reaction is purified following in vitro transcription. In preferred embodiments, this dsRNA serves as an adjuvant when sensed by dsRNA sensors including MDA-5, RIG-I, TLR-3.

In other embodiments, mRNA concentration is adjusted for both peak antigen expression and for peak triggering of TLR-7/8 since the ssRNA is itself sensed.

Oil-Emulsion Adjuvants include squalene-water emulsions, such as MF59 (5% Squalene, 0.5% Tween 80, and 0.5% Span 85, formulated into submicron particles using a microfluidizer). See, e.g., WO90/14837 and Podda, Vaccine 19: 2673-2680, 2001. Additional adjuvants for use in the compositions are submicron oil-in-water emulsions. Examples of submicron oil-in-water emulsions for use herein include squalene/water emulsions optionally containing varying amounts of MTP-PE, such as a submicron oil-in-water emulsion containing 4-5% w/v squalene, 0.25-1.0% w/v Tween 80 (polyoxyelthylenesorbitan monooleate), and/or 0.25-1.0% Span 85 (sorbitan trioleate), and, optionally, N-acetylmuramyl-L-alanyl-D-isogluatminyl-L-alanine-2-(1′-2′-dipalmitoyl-s- -n-glycero-3-huydroxyphosphophoryloxy)-ethylamine (MTP-PE), for example, the submicron oil-in-water emulsion known as “MF59” (International Publication No. WO90/14837; U.S. Pat. Nos. 6,299,884 and 6,451,325, incorporated herein by reference in their entirety. MF59 can contain 4-5% w/v Squalene (e.g., 4.3%), 0.25-0.5% w/v Tween 80, and 0.5% w/v Span 85 and optionally contains various amounts of MTP-PE, formulated into submicron particles using a microfluidizer such as Model 110Y microfluidizer (Microfluidics, Newton, Mass.). For example, MTP-PE can be present in an amount of about 0-500 μg/dose, or 0-250 μg/dose, or 0-100 μg/dose. Submicron oil-in-water emulsions, methods of making the same and immunostimulating agents, such as muramyl peptides, for use in the compositions, are described in detail in International Publication No. WO90/14837 and U.S. Pat. Nos. 6,299,884 and 6,451,325.

Complete Freund's adjuvant (CFA) and incomplete Freund's adjuvant (IFA) can also be used as adjuvants in the invention.

Saponin Adjuvant Formulations can also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants.

Saponin can also be commercially obtained from Smilax ornata (sarsaprilla), Gypsophilla paniculata (brides veil), and Saponaria officianalis (soap root). Saponin adjuvant formulations can include purified formulations, such as QS21, as well as lipid formulations, such as Immunostimulating Complexes (ISCOMs; see below). Saponin compositions have been purified using High Performance Thin Layer Chromatography (HPLC) and Reversed Phase High Performance Liquid Chromatography (RP-HPLC). Specific purified fractions using these techniques have been identified, including QS7, QS17, QS18, QS21, QH-A, QH-B and QH-C. A method of production of QS21 is disclosed in U.S. Pat. No. 5,057,540. Saponin formulations can also comprise a sterol, such as cholesterol (see WO96/33739). Combinations of saponins and cholesterols can be used to form unique particles called ISCOMs. ISCOMs typically also include a phospholipid such as phosphatidylethanolamine or phosphatidylcholine. Any known saponin can be used in ISCOMs. For example, an ISCOM can include one or more of Quil A, QHA and QHC. ISCOMs are described in EP0109942, WO96/11711, and WO96/33739. Optionally, the ISCOMS can be devoid of additional detergent. See WO00/07621. A description of the development of saponin based adjuvants can be found at Barr, et al., “ISCOMs and other saponin based adjuvants”, Advanced Drug Delivery Reviews 32: 247-27, 1998. See also Sjolander, et al., “Uptake and adjuvant activity of orally delivered saponin and ISCOM vaccines”, Advanced Drug Delivery Reviews 32: 321-338, 1998.

Bioadhesives and mucoadhesives can also be used as adjuvants. Suitable bioadhesives can include esterified hyaluronic acid microspheres (Singh et al., J. Cont. Rel. 70:267-276, 2001) or mucoadhesives such as cross-linked derivatives of poly(acrylic acid), polyvinyl alcohol, polyvinyl pyrollidone, polysaccharides and carboxymethylcellulose. Chitosan and derivatives thereof can also be used as adjuvants in the invention disclosed for example in WO99/27960.

Adjuvant Microparticles: Microparticles can also be used as adjuvants. Microparticles (i.e., a particle of about 100 nm to about 150 μm in diameter, or 200 nm to about 30 μm in diameter, or about 500 nm to about 10 μm in diameter) formed from materials that are biodegradable and/or non-toxic (e.g., a poly(alpha-hydroxy acid), a polyhydroxybutyric acid, a polyorthoester, a polyanhydride, a polycaprolactone, and the like), with poly(lactide-co-glycolide) are envisioned, optionally treated to have a negatively-charged surface (e.g., with SDS) or a positively-charged surface (e.g., with a cationic detergent, such as CTAB).

Examples of liposome formulations suitable for use as adjuvants are described in U.S. Pat. Nos. 6,090,406, 5,916,588, and EP 0 626 169.

Additional adjuvants include polyoxyethylene ethers and polyoxyethylene esters. WO99/52549. Such formulations can further include polyoxyethylene sorbitan ester surfactants in combination with an octoxynol (WO 01/21207) as well as polyoxyethylene alkyl ethers or ester surfactants in combination with at least one additional non-ionic surfactant such as an octoxynol (WO 01/21152). In some embodiments, polyoxyethylene ethers can include: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, or polyoxyethylene-23-lauryl ether.

PCPP formulations for use as adjuvants are described, for example, in Andrianov et al., Biomaterials 19: 109-115, 1998.1998. Examples of muramyl peptides suitable for use as adjuvants in the invention can include N-acetyl-muramyl-L-threonyl-D-isoglutamine (thr-MDP), N-acetyl-normuramyl-l-alanyl-d-isoglutamine (nor-MDP), and N-acetylmuramyl-1-alanyl-d-isoglutaminyl-1-alanine-2-(1′-2′-dipalmitoyl-s- -n-glycero-3-hydroxyphosphoryloxy)-ethylamine MTP-PE). Examples of imidazoquinolone compounds suitable for use as adjuvants in the invention can include Imiquimod and its homologues, described further in Stanley, “Imiquimod and the imidazoquinolones: mechanism of action and therapeutic potential” Clin Exp Dermatol 27: 571-577, 2002 and Jones, “Resiquimod 3M”, Curr Opin Investig Drugs 4: 214-218, 2003. Human immunomodulators suitable for use as adjuvants in the invention can include cytokines, such as interleukins (e.g., IL-1, IL-2, IL-4, IL-5, IL-6, IL-7, IL-12, and the like), interferons (e.g., interferon-gamma), macrophage colony stimulating factor, and tumor necrosis factor.

### E. Pharmaceutically Acceptable Carriers

In some embodiments, the compositions include one or more pharmaceutically acceptable carriers, or excipients, or preservatives. Pharmaceutically acceptable carriers include compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the Food and Drug Administration. Pharmaceutically acceptable carriers include, but are not limited to, buffers, diluents, preservatives, binders, stabilizers, a mixture, or polymer of sugars (lactose, sucrose, dextrose, etc.), salts, and combinations thereof.

The compositions may be administered in combination with one or more physiologically or pharmaceutically acceptable carriers, thickening agents, co-solvents, adhesives, antioxidants, buffers, viscosity, and absorption enhancing agents and agents capable of adjusting osmolarity of the formulation. Proper formulation is dependent upon the route of administration chosen. If desired, the compositions may also contain minor amounts of nontoxic auxiliary substances such as wetting or emulsifying agents, dyes, pH buffering agents, or preservatives.

In general, pharmaceutical compositions are provided including effective amounts of the composition, and optionally include pharmaceutically acceptable diluents, preservatives, solubilizers, emulsifiers, adjuvants and/or carriers. Such compositions include diluents such as sterile water, buffered saline of various buffer content (e.g., Tris-HCl, acetate, phosphate), pH and ionic strength; and optionally, additives such as antioxidants (e.g., ascorbic acid, sodium metabisulfite), and preservatives and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate.

In some embodiments, the pharmaceutical composition is a saline solution, preferably a buffered saline solution phosphate buffered saline or sterile saline, or tissue culture medium.

### 1. Formulations

The disclosed compositions can be formulated in a pharmaceutical composition. Pharmaceutical compositions including antigens, adjuvants, and the combination thereof are provided. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration.

In some embodiments, the compositions are administered systemically, for example, by intravenous or intraperitoneal administration, in an amount effective for delivery of the compositions to targeted cells.

Most typically, the compositions are administered by intramuscular, intradermal, subcutaneous injection or infusions, or intravenous injection or infusion, or by intranasal delivery.

Compositions and pharmaceutical formulations thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), antioxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria-retaining filter, by incorporating sterilizing agents into the compositions, by irradiating the compositions, or by heating the compositions.

### F. Immunogenic Compositions and Vaccines

Immunogenic compositions and vaccines are also provided. Typically, an immunogenic composition includes an adjuvant, an antigen (which may be e.g., a nucleic acid encoding one or more viral proteins, e.g., including but not limited to viral proteins of MCPyV such as LTA protein, a variant, or an immunogenic domain or fragment thereof), or a combination thereof. The combination of an adjuvant and an antigen can be referred to as a vaccine. When administered to a subject in combination, the adjuvant and antigen can be administered in separate pharmaceutical compositions, or they can be administered together in the same pharmaceutical composition. The nucleic acids encoding the one or more viral proteins (e.g., mRNA) can serve as the antigen component of an immunogenic composition or vaccine formulation. Thus, in some embodiments, the composition includes both an antigen and an adjuvant. Two or more different antigens, one or more different adjuvants, or combinations thereof, can be used or combined. In particular embodiments, the formulation is a nanoparticle-based vaccines with or without adjuvant and using mRNA or DNA as the means of delivering the antigen.

### III. Methods of Making Immunogenic Compositions

Methods of making an isolated nucleic acid encoding a recombinant protein derived from a virus are described. Methods of making mRNA molecules encoding one or more viral antigens expressed in virally driven cancers are described. In some embodiments, the nucleic acid construct includes nucleotide sequence encoding viral antigens, and/or immunogenic domains and fragments thereof, and optionally one or more of (i) signal peptide, (ii) one or more restriction sites, (iii) promoter region such as T7 promoter, (iv) TRILINK CAP site, (v) traditional KOZAK sequence, (vi) 5′ untranslated region (UTR), (vii) 3′ UTR, and (viii) poly(A) tail. In other embodiments, the construct also includes a sequence encoding a purification/affinity tag such as FLAG tag. In some embodiments, the viral antigens include those derived from a truncated form of the viral Large T Antigen (LTA) of Merkel Cell Polyomavirus (MCPyV), or another antigen discussed herein.

In general, nucleic acid constructs include a regulatory sequence operably linked to a nucleotide sequence encoding a recombinant protein including the viral antigens as disclosed. Regulatory sequences (or expression control sequences) typically do not encode a gene product, but instead affect the expression of the nucleic acid sequences to which they are operably linked. The nucleotide sequences encoding the recombinant protein are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. The vector is preferably an expression vector in which the DNA sequence encoding the recombinant protein is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g., transcription initiates in promoter and proceeds through the DNA sequence coding for the recombinant protein. In some embodiments, the expression vector includes a promoter capable of directing the transcription of a cloned gene or cDNA. The promoter may be any DNA sequence, which shows transcriptional activity in the host cell of choice and may be derived from genes encoding proteins either homologous or heterologous to the host cell. Exemplary promoters include a T7 promoter.

The disclosed antigens can be provided (e.g., administered to a subject in need thereof) in numerous ways, e.g., as DNA or RNA construct (e.g., a viral vector), as mRNA, or as expressed protein.

Particularly preferred embodiments are exemplified in the Examples and in the disclosed compositions. In some embodiments, mRNA constructs are produced from purified, double stranded, linearized template DNAs. mRNA can be prepared by in vitro expression and are harvested for use in the disclosed methods. In one embodiment, mRNA constructs are produced from purified, double stranded, linearized template DNA via commercially available kit such as HISCRIBE™ T7 High Yield RNA Kit (New England Biolabs).

Alternatively, antigenic protein can be expressed in host cells, isolated, and administered to a subject in need thereof.

### IV. Methods of Making Dendritic Cells and T Cell Therapeutics

Methods of making T cell therapeutics specific to one or more viral antigens are described. In some embodiments, methods for profiling viral and/or tumor antigen-specific T cell receptor (TCR) repertoires are also described.

In some embodiments, immune cells are isolated from blood or tumor. The immune cells can be T cells and/or dendritic cells. In one embodiment, immune cells such as T cells and/or dendritic cells are isolated from peripheral blood mononuclear cells (PBMC). In another embodiment, immune cells such as T cells and/or dendritic cells are from leukopaks collected via leukapheresis. In some embodiments, T cells are enriched by binding of a ligand to T cell specific markers. In some embodiments, the markers may be CD3, CD4, CD8, CD28, or any combination therewith. In some embodiments, the ligands are antibodies. In one embodiment the antibodies are conjugated to beads. In one embodiment, the antibodies are fluorescently labeled. In one embodiment, the cells are separated by cell sorting. In some embodiments, T cells are cultured in the presence of cytokines such as IL-2, IL-7, and/or IL-15 for a sufficient amount of time to enrich cultures for T cells, for example, 1 day, 2 days, 3 days, 4 days, 5 days, 6 days, 1 week, 2 weeks, 3 weeks, 4 weeks, or more than 4 weeks.

As shown in the Examples, the mRNA vaccine can be introduced into monocyte-derived dendritic cells (Mo-DCs) and these DCs can be used to stimulate T cells repeatedly to enrich for vaccine-specific T cell clonotypes. Thus, in some embodiments, monocyte-derived dendritic cells (Mo-DC) are isolated (e.g., using a CD14+ magnetic selection kit) and enriched from a patient with a particular viral infection of interest (e.g., MCPyV) or a virally driven cancer (e.g., MCC). In one embodiment, Mo-DC cultured in GM-CSF and IL-4 after enrichment. The disclosed immunogenic compositions can be introduced into Mo-DC, for example via electroporation or using lipofectamine. The expression of the immunogenic compositions can be verified, for example, using Western blotting analysis. In some embodiments, immunogenic composition-treated dendritic cells or Mo-DCs are used as a part of an adoptive cell therapy that includes administering the treated cells to a subject.

In some embodiments, T cells are stimulated with Mo-DCs expressing the desired viral antigen(s) for those T cells with specificity towards the viral antigen(s) introduced into the Mo-DC. Generally, T cells are stimulated enough times with the Mo-DC expressing the desired viral antigen(s), for example once, twice, three times, four times, five times, more than five times, to provide a desired amount of T cells. Stimulation can be ex vivo, e.g., to create T cells for adoptive therapy, or can be in vivo, e.g., as a result of dendritic cells adoptive therapy.

In further embodiments, T cells that are specific towards the desired viral antigen(s) are isolated and TCRs from individual T cells are identified. In some embodiments, single T cells are sequenced. In some embodiments, single T cells are diluted such that each well of a plate contains a single cell. In some embodiments, the single T cells are expanded in tissue culture. In some embodiments, the nucleic acid from the single expanded T cell clones is sequenced. In some embodiments, the nucleic acid from the single cells is sequenced without expanding the cells.

In preferred embodiments, a subject in need thereof is treated based on the TCR repertoire derived from the same or different subject. In one embodiment, a virally derived tumor antigen vaccine is selected based on the TCRs. In another embodiment, a subject is treated with T cells expressing one or more TCRs specific to a virally derived tumor antigen. The ability to effectively profile the TCR repertoire and to link individual T cells containing specific TCRs to an epitope thereby provides an important approach to the identification of T cell targets useful for therapy. Once identified, such TCRs provide molecular reagents to prove the functionality of epitope-specific T cells against tumor targets and to follow highly specific T cells longitudinally in a patient and also facilitate adoptive therapy with T cells engineered to contain these epitope-specific TCRs. In further embodiments, identified TCRs are affinity matured to recognize their cognate antigen to provide enhanced sensitivity and specificity to the response.

In further embodiments, an immunogenic composition or vaccine is selected based on the TCRs identified. In one embodiment, identification of the T cell repertoire and testing in functional assays is used to determine an immunogenic composition or vaccine to be administered to a subject in need thereof. In one embodiment, the peptide antigens are selected based on the binding affinity of the peptide to a TCR. In one embodiment, the selecting is based on a combination of both the quantity and the binding affinity. In other embodiments, a TCR that binds strongly to a virally derived tumor antigen in a functional assay, but that is not highly represented in the TCR repertoire of a subject represents a good candidate for a vaccine.

In some embodiments, the methods further involve adoptive transfer of T cells, specific for selected antigens, such as tumor associated antigens. Selected TCRs can be cloned, and nucleic acids encoding the TCR can be transfected into T cells such that the desired TCR is expressed by the cells that are transferred. In some embodiments, chimeric antigen receptors (CARs) are used in order to generate immunoresponsive cells, such as T cells, specific for selected targets.

Methods of adoptive dendritic cells and T cell therapy are known in the art and used in clinical practice. See, e.g., Abakushina, et al., Vaccines (Basel). 2021 November; 9(11): 1363 doi: 10.3390/vaccines9111363. Generally adoptive T cell therapy involves the isolation and ex vivo expansion of tumor specific T cells to achieve greater number of T cells than what could be obtained by vaccination alone. The tumor specific T cells are then infused into patients with cancer in an attempt to give their immune system the ability to overwhelm remaining tumor via T cells which can attack and kill cancer. Several forms of adoptive T cell therapy can be used for cancer treatment including, but not limited to, culturing tumor infiltrating lymphocytes or TIL; isolating and expanding one particular T cell or clone; and using T cells that have been engineered to recognize and attack tumors. In the methods, the disclosed antigenic compositions are used to prime the T cells. In some embodiments, the T cells are taken directly from the patient's blood after they have received treatment or immunization with the composition. Methods of priming and activating T cells in vitro for adaptive T cell cancer therapy are known in the art. See, for example, Wang, et al., Blood, 109(11):4865-4872 (2007) and Hervas-Stubbs, et al., J. Immunol., 189(7):3299-310 (2012). The methods can be used in conjunction with disclosed antigenic compositions to prime and activate T cells against the cancer.

Historically, adoptive T cell therapy strategies have largely focused on the infusion of tumor antigen specific cytotoxic T cells (CTL) which can directly kill tumor cells. However, CD4+T helper (Th) cells and Natural Killer (NK) cells can also be used. Th cells can activate antigen-specific effector cells and recruit cells of the innate immune system such as macrophages and dendritic cells to assist in antigen presentation (APC), and antigen primed Th cells can directly activate tumor antigen-specific CTL. As a result of activating APC, antigen specific Th1 have been implicated as the initiators of epitope or determinant spreading which is a broadening of immunity to other antigens in the tumor. The ability to elicit epitope spreading broadens the immune response to many potential antigens in the tumor and can lead to more efficient tumor cell kill due to the ability to mount a heterogeneic response. In this way, adoptive T cell therapy can used to stimulate endogenous immunity.

### V. Exemplary T Cell Receptor Sequence, and Engineered TCRs and T Cell Made Therewith

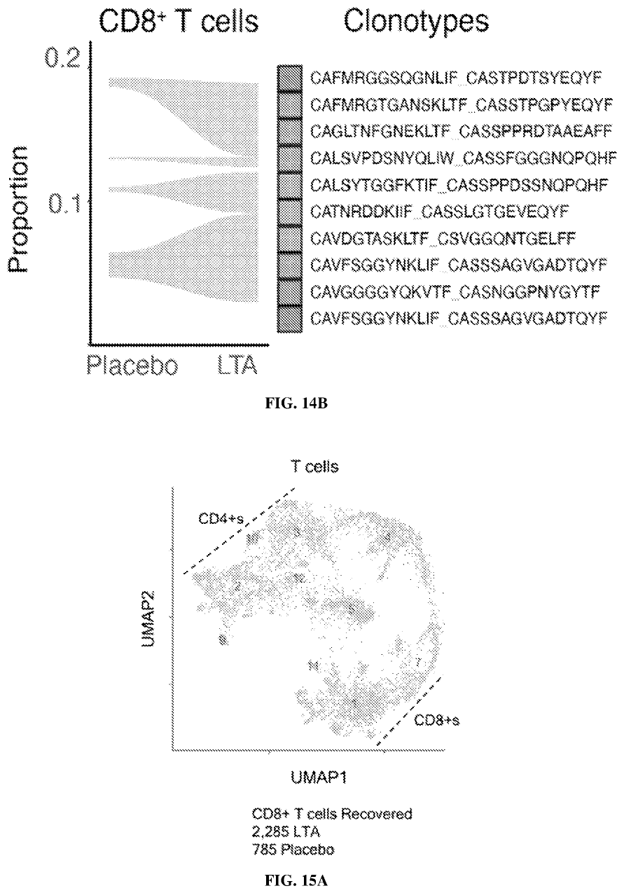

The Examples below, and FIG. 14B in particular, illustrates amino acid sequences of TCR alpha and beta chain hypervariable regions corresponding to CDR3a and CDR3b separated by a dash, also provided here in Table 1, that were selected as clonotypes of MCC antigen-specific TCRs.

TABLE 1
Exemplary TCR Sequences

| TCR alpha chain | TCR beta chain |
|---|---|
| CAFMRGGSQGNLIF | CASTPDTSYEQYF |
| (SEQ ID NO: 27) | (SEQ ID NO: 28) |
| CAFMRGTGANSKLIF | CASSTPGPYEQYF |
| (SEQ ID NO: 29) | (SEQ ID NO: 30) |
| CAGLTNFGNEKLTF | CASSPPRDTAAEAFF |
| (SEQ ID NO: 31) | (SEQ ID NO: 32) |
| CALSVPDSNYQLIW | CASSFGGGNQPQHF |
| (SEQ ID NO: 33) | (SEQ ID NO: 34) |
| CALSYTGGFKTIF | CASSPPDSSNQPQHF |
| (SEQ ID NO: 35) | (SEQ ID NO: 36) |
| CATNRDDKIIF | CASSLGTGEVEQYF |
| (SEQ ID NO: 37) | (SEQ ID NO: 38) |
| CAVDGTASKLTF | CSVGGQNTGELFF |
| (SEQ ID NO: 39) | (SEQ ID NO: 40) |
| CAVFSGGYNKLIF | CASSSAGVGADTQYF |
| (SEQ ID NO: 41) | (SEQ ID NO: 42) |
| CAVGGGGYQKVTF | CASNGGPNYGYTF |
| (SEQ ID NO: 43) | (SEQ ID NO: 44) |
| CAVFSGGYNKLIF | CASSSAGVGADTOYF |
| (SEQ ID NO: 45) | (SEQ ID NO: 46) |

These are the specific sequences of the unique binding regions of the TCRs. These expanded clonotypes are predicted to have affinity for the LTA antigen epitope—HLA class 1 complex presented by dendritic cells. TCRs including these sequences can be used to create and validate T cell therapies such as engineered TCR-T cells against LTA-HLA class 1 complexes on MCC tumor cells.

Thus, sequences for engineered TCR or CAR including the provided sequences, nucleic acids encoding the same, and cells harboring and/or expressing the nucleic acids and/or TCR or CAR proteins are provided, and can be used in the adoptive T cell compositions and method disclosed herein.

As used herein, the term “T cell receptor” (TCR) refers to an immunoglobulin superfamily member having a variable binding domain, a constant domain, a transmembrane region, and a short cytoplasmic tail (see, e.g., Janeway et al., Immunobiology: The Immune System in Health and Disease, 3.sup.rd Ed., Current Biology Publications, p. 4:33, 1997, relevant portions incorporated herein by reference) capable of specifically binding to an antigen peptide bound to, or presented by an MHC. A TCR can be found on the surface of a cell or in soluble form and generally is composed of a heterodimer having α and β chains (also known as TCRα and TCRβ, respectively), or γ and δ chains (also known as TCRγ and TCRδ, respectively). Like immunoglobulins, the extracellular portion of TCR chains (e.g., α-chain, β-chain) contain two immunoglobulin domains: a variable domain (e.g., α-chain variable domain or Vα, β-chain variable domain or Vβ; typically amino acids 1 to 116 based on Kabat numbering (Kabat et al., “Sequences of Proteins of Immunological Interest, US Dept. Health and Human Services, Public Health Service National Institutes of Health, 1991, 5.sup.th ed., relevant portions incorporated herein by reference) at the N-terminus; and one constant domain (e.g., α-chain constant domain or Cα, typically amino acids 117 to 259 based on Kabat, β-chain constant domain or Cβ, typically amino acids 117 to 295 based on Kabat) adjacent to the cell membrane. Also, like immunoglobulins, the variable domains contain complementary determining regions (CDRs) separated by framework regions (FRs) (see, e.g., Jones et al., Proc. Nat'l Acad. Sci. U.S.A. 87:9138, 1990; Chothia et al., EMBO 1 7:3745, 1988; see also Lefranc et al., Dev. Comp. Immunol. 27:55, 2003, relevant portions incorporated herein by reference). TCR variable domain sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300, relevant portions incorporated herein by reference). A numbering scheme provides a standardized delineation of framework regions and CDRs in the TCR variable domains.

For example, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. As used herein, the term “TCR” should be understood to also include functional TCR fragments thereof. The term also encompasses intact or full-length TCRs, including TCRs in the ap form or γδ form when having the CDR1,2, and/or 3. In some embodiments, the provided sequences are the CDR3.

It is known that the variable domains of the TCR chains associate to form complementarity determining regions (CDRs) analogous to immunoglobulins, which confer antigen recognition and determine peptide specificity by forming the binding site of the TCR molecule, determine peptide specificity, and determine the MHC molecules that forms the peptide-MHC complex. Like immunoglobulins, TCR CDRs are separated by framework regions (FRs) (see, e.g., Jores et al., PNAS U.S.A. 87:9138, 1990; Chothia et al., EMBO J. 7:3745, 1988; see also Lefranc et al., Dev. Comp. Immunol. 27:55, 2003 which is specifically incorporated herein by reference in its entirety. In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the f-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5.sup.th ed.) at the N-terminus, and one constant domain (e.g., α-chain constant domain or Ca, typically amino acids 117 to 259 based on Kabat, β-chain constant domain or Cp, typically amino acids 117 to 295 based on Kabat) adjacent to the cell membrane, relevant portions incorporated herein by reference. Generally, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond linking the two chains. In certain examples, the TCR may have an additional cysteine residue in each of the α and β chains such that the TCR contains two disulfide bonds in the constant domains.

Generally, TCR chains contain a transmembrane domain, although that can be removed, or replaced with other transmembrane domain(s). Often, the transmembrane domain is positively charged. Generally, TCR contains a cytoplasmic tail, although that can be removed, or replaced with other cytoplasmic tail(s). Generally, the structure allows the TCR to associate with other molecules of the CD3 complex. For example, the TCR containing constant domains with a transmembrane region can anchor the protein in the cell membrane and associate with invariant subunits of the CD3 signaling complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell.

Generally, the TCR may be a heterodimer of two chains α and β (or γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T-cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15: 169-180 and Cohen et al. (2005) J Immunol. 175:5799-5808). In some embodiments, phage display is used to isolate TCRs against a target antigen (see, e.g., Varela-Rohena et al. (2008) Nat Med. 14: 1390-1395 and Li (2005) Nat Biotechnol. 23:349-354, relevant portions incorporated herein by reference). In some examples, the TCR or antigen-binding portion thereof is synthetically generated from knowledge of the sequence of the TCR.

Exemplary TCRs including a TCR alpha chain variable domain including an amino acid sequence of Table 1, or a variant thereof with at least 70%, 75%, 80%, 85%, 90%, or 95% sequence identity thereto, in combination with a TCR beta chain variable domain including an amino acid sequence of Table 1, or a variant thereof with at least 70%, 75%, 80%, 85%, 90%, or 95% sequence identity thereto are provided.

In some embodiments, the TCR includes a TCR alpha chain variable domain including the amino acid sequence:
CAFMRGGSQGNLIF (SEQ ID NO:27) or a variant thereof with at least 70%, 75%, 80%, 85%, 90%, or 95% sequence identity thereto,
CAFMRGTGANSKLTF (SEQ ID NO:29) or a variant thereof with at least 70%, 75%, 80%, 85%, 90%, or 95% sequence identity thereto,
CAGLTNFGNEKLTF (SEQ ID NO:31) or a variant thereof with at least 70%, 75%, 80%, 85%, 90%, or 95% sequence identity thereto,
CALSVPDSNYQLIW (SEQ ID NO:33) or a variant thereof with at least 70%, 75%, 80%, 85%, 90%, or 95% sequence identity thereto,
CALSYTGGFKTIF (SEQ ID NO:35) or a variant thereof with at least 70%, 75%, 80%, 85%, 90%, or 95% sequence identity thereto,
CATNRDDKIIF (SEQ ID NO:37) or a variant thereof with at least 70%, 75%, 80%, 85%, 90%, or 95% sequence identity thereto,
CAVDGTASKLTF (SEQ ID NO:39) or a variant thereof with at least 70%, 75%, 80%, 85%, 90%, or 95% sequence identity thereto,
CAVFSGGYNKLIF (SEQ ID NO:41) or a variant thereof with at least 70%, 75%, 80%, 85%, 90%, or 95% sequence identity thereto,
CAVGGGGYQKVTF (SEQ ID NO:43) or a variant thereof with at least 70%, 75%, 80%, 85%, 90%, or 95% sequence identity thereto, or
CAVFSGGYNKLIF (SEQ ID NO:45) or a variant thereof with at least 70%, 75%, 80%, 85%, 90%, or 95% sequence identity thereto;
in combination with a TCR beta chain variable domain including the amino acid sequence:
CASTPDTSYEQYF (SEQ ID NO:28) or a variant thereof with at least 70%, 75%, 80%, 85%, 90%, or 95% sequence identity thereto;
CASSTPGPYEQYF (SEQ ID NO:30) or a variant thereof with at least 70%, 75%, 80%, 85%, 90%, or 95% sequence identity thereto;
CASSPPRDTAAEAFF (SEQ ID NO:32) or a variant thereof with at least 70%, 75%, 80%, 85%, 90%, or 95% sequence identity thereto;
CASSFGGGNQPQHF (SEQ ID NO:34) or a variant thereof with at least 70%, 75%, 80%, 85%, 90%, or 95% sequence identity thereto;
CASSPPDSSNQPQHF (SEQ ID NO:36) or a variant thereof with at least 70%, 75%, 80%, 85%, 90%, or 95% sequence identity thereto;
CASSLGTGEVEQYF (SEQ ID NO:38) or a variant thereof with at least 70%, 75%, 80%, 85%, 90%, or 95% sequence identity thereto;
CSVGGQNTGELFF (SEQ ID NO:40) or a variant thereof with at least 70%, 75%, 80%, 85%, 90%, or 95% sequence identity thereto;
CASSSAGVGADTQYF (SEQ ID NO:42) or a variant thereof with at least 70%, 75%, 80%, 85%, 90%, or 95% sequence identity thereto;
CASNGGPNYGYTF (SEQ ID NO:44) or a variant thereof with at least 70%, 75%, 80%, 85%, 90%, or 95% sequence identity thereto; or
CASSSAGVGADTQYF (SEQ ID NO:46) or a variant thereof with at least 70%, 75%, 80%, 85%, 90%, or 95% sequence identity thereto.

In some embodiments, the TCR includes a TCR alpha chain variable domain including the amino acid sequence of SEQ ID NO:27 or a variant thereof with at least 70%, 75%, 80%, 85%, 90%, or 95% sequence identity thereto, in combination with a TCR beta chain variable domain including the amino acid sequence of SEQ ID NO:28 or a variant thereof with at least 70%, 75%, 80%, 85%, 90%, or 95% sequence identity thereto.

In some embodiments, the TCR includes a TCR alpha chain variable domain including the amino acid sequence of SEQ ID NO:29 or a variant thereof with at least 70%, 75%, 80%, 85%, 90%, or 95% sequence identity thereto, in combination with a TCR beta chain variable domain including the amino acid sequence of SEQ ID NO:30 or a variant thereof with at least 70%, 75%, 80%, 85%, 90%, or 95% sequence identity thereto.

In some embodiments, the TCR includes a TCR alpha chain variable domain including the amino acid sequence of SEQ ID NO:31 or a variant thereof with at least 70%, 75%, 80%, 85%, 90%, or 95% sequence identity thereto, in combination with a TCR beta chain variable domain including the amino acid sequence of SEQ ID NO:32 or a variant thereof with at least 70%, 75%, 80%, 85%, 90%, or 95% sequence identity thereto.

In some embodiments, the TCR includes a TCR alpha chain variable domain including the amino acid sequence of SEQ ID NO:33 or a variant thereof with at least 70%, 75%, 80%, 85%, 90%, or 95% sequence identity thereto, in combination with a TCR beta chain variable domain including the amino acid sequence of SEQ ID NO:34 or a variant thereof with at least 70%, 75%, 80%, 85%, 90%, or 95% sequence identity thereto.

In some embodiments, the TCR includes a TCR alpha chain variable domain including the amino acid sequence of SEQ ID NO:35 or a variant thereof with at least 70%, 75%, 80%, 85%, 90%, or 95% sequence identity thereto, in combination with a TCR beta chain variable domain including the amino acid sequence of SEQ ID NO:36 or a variant thereof with at least 70%, 75%, 80%, 85%, 90%, or 95% sequence identity thereto.

In some embodiments, the TCR includes a TCR alpha chain variable domain including the amino acid sequence of SEQ ID NO:37 or a variant thereof with at least 70%, 75%, 80%, 85%, 90%, or 95% sequence identity thereto, in combination with a TCR beta chain variable domain including the amino acid sequence of SEQ ID NO:38 or a variant thereof with at least 70%, 75%, 80%, 85%, 90%, or 95% sequence identity thereto.

In some embodiments, the TCR includes a TCR alpha chain variable domain including the amino acid sequence of SEQ ID NO:39 or a variant thereof with at least 70%, 75%, 80%, 85%, 90%, or 95% sequence identity thereto, in combination with a TCR beta chain variable domain including the amino acid sequence of SEQ ID NO:40 or a variant thereof with at least 70%, 75%, 80%, 85%, 90%, or 95% sequence identity thereto.

In some embodiments, the TCR includes a TCR alpha chain variable domain including the amino acid sequence of SEQ ID NO:41 or a variant thereof with at least 70%, 75%, 80%, 85%, 90%, or 95% sequence identity thereto, in combination with a TCR beta chain variable domain including the amino acid sequence of SEQ ID NO:42 or a variant thereof with at least 70%, 75%, 80%, 85%, 90%, or 95% sequence identity thereto.

In some embodiments, the TCR includes a TCR alpha chain variable domain including the amino acid sequence of SEQ ID NO:43 or a variant thereof with at least 70%, 75%, 80%, 85%, 90%, or 95% sequence identity thereto, in combination with a TCR beta chain variable domain including the amino acid sequence of SEQ ID NO:44 or a variant thereof with at least 70%, 75%, 80%, 85%, 90%, or 95% sequence identity thereto.

In some embodiments, the TCR includes a TCR alpha chain variable domain including the amino acid sequence of SEQ ID NO:45 or a variant thereof with at least 70%, 75%, 80%, 85%, 90%, or 95% sequence identity thereto, in combination with a TCR beta chain variable domain including the amino acid sequence of SEQ ID NO:46 or a variant thereof with at least 70%, 75%, 80%, 85%, 90%, or 95% sequence identity thereto.

The CDR3 is the portion of TCR receptors that is most involved in interactions with intact soluble antigens (B cells) or intracellular processed antigens presented as immunogenic peptides loaded in MHC molecules (T cells). See, e.g., Yassai, et al., Immunogenetics. 2009 July; 61(7): 493-502. Published online 2009 Jul. 1. doi: 10.1007/s00251-009-0383-x which is specifically incorporated by reference herein in its entirety. In preferred embodiments, the variable domains of the alpha and beta chains of the TCR each include CDR1, CDR2, and CDR3 complementarity determining regions, and the provided sequences are found in the CDR3 region of the variable sequence.

Thus, the disclosed sequences can be substituted for the CDR3 sequences of any known TCR to produce the provided an engineered TCR. Methods of making and using TCRs and reference sequences are known in the art. See, for example, Shafer, et al., Cancer Therapy With TCR-Engineered T Cells: Current Strategies, Challenges, and Prospects, Front. Immunol., Sec. T Cell Biology Volume 13-2022, doi.org/10.3389/fimmu.2022.835762, US Published Application Nos. 20230399402, and 2023/0398217, each of which is specifically incorporated by reference herein in its entirety.

Pharmaceutical compositions and formulations including an effective amount of TCR-engineered cells, e.g., T cells, are also provided, as are methods of using them to treat the subject provided herein, e.g., via adoptive T cell therapy as discussed in more detail elsewhere herein. In particular embodiments, the subject has MCC.

### VI. Methods of Use

The disclosed compositions can be administered as part of prophylactic vaccines or immunogenic compositions which confer resistance in a subject to subsequent exposure to infectious agents, or as part of therapeutic vaccines, which can be used to initiate or enhance a subject's immune response to a pre-existing antigen, such as a virally derived tumor antigen in a subject with cancer.

### A. Methods of Treatment

Methods of inducing an immune response in a subject (e.g., a human) by administering to the subject a therapeutically effective amount of a disclosed immunogenic or vaccine composition are provided. The immune response can be induced, increased, or enhanced by the composition compared to a control (e.g., absence of the composition or presence of another composition). The composition can include an effective amount of isolated nucleic acid sequences (e.g., a viral vector, mRNA) encoding one or more viral antigens of one or more viruses that cause cancer, and/or immunogenic domains and fragments thereof.

Adjuvant can optionally be delivered together or separately.

In some embodiments, the compositions induce an effector cell response such as a CD4⁺ or CD8⁺ T-cell immune response, against at least one of the component antigen(s) or antigenic compositions compared to the effector cell response obtained under control conditions (e.g., absence of the composition or presence of another composition). The term “improved effector cell response” refers to a higher effector cell response such as a CD8 or CD4 response obtained in a subject after administration of a disclosed composition than that obtained under control conditions.

In some embodiments, the disclosed compositions induce CD4 or CD8 T-cell immune response with increased activation and the generation of memory phenotypes. In some embodiments, the disclosed composition is administered to a subject in need thereof in an effective amount to induce increased expression of PD-1, and/or increased expression of CD45RO on the surface of CD8+ T cells. In other embodiments, the disclosed composition is administered to a subject in need thereof in an effective amount to induce increased CD8 T-cell effector cell function such as enhanced cytokine expression, e.g., IFN-γ.

In other embodiments, the subject is administered a therapeutically effective amount of dendritic cells or T cells primed or engineered according to the disclosed compositions and methods and/or engineered to include a disclose TCR sequence (i.e., adoptive T cell therapy).

All the methods can include the step of identifying and selecting a subject in need of treatment, or a subject who would benefit from administration with the described compositions.

### B. Methods of Administration

The compositions are generally administered to a subject in an effective amount. As used herein the term “effective amount” means a dosage sufficient to inhibit, or prevent one or more infections, or symptoms of a disease or to otherwise provide a desired pharmacologic and/or physiologic effect. The precise dosage will vary according to a variety of factors such as subject-dependent variables (e.g., age, immune system health, etc.), the specific variant of virus, and the treatment being affected.

The pharmaceutical compositions can be for administration by parenteral (intramuscular, intraperitoneal, intravenous, or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration.

In some embodiments, the compositions are administered locally, for example by intranasal administration. Typically, local administration causes an increased localized concentration of the compositions which is greater than that which can be achieved by systemic administration. In some embodiments, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can affect a sustained release of the particles to the immediate area of the implant.

In one embodiment, the method includes administration via a nebulizer to a subject of an effective amount of the disclosed composition.

The disclosed immunogenic compositions, cells, and pharmaceutical formulations thereof can be used alone or in combination other interventions. In some embodiments, the subject has advanced, inoperable cancer and/or metastases. In some embodiments, the immunogenic composition, cells, or pharmaceutical formulation is administered in combination with or second therapeutic intervention such as conventional antiviral and/or anticancer therapeutics, or procedures for example, radiation or surgery. In particular embodiments, the immunogenic compositions, cells, and pharmaceutical formulations is administered to a subject a with a virally driven cancer as an adjunct to surgery or radiation, e.g., before, during, and/or after surgery or radiation. For example, in some embodiments, the subject first receives surgery and/or radiation to remove one or more tumors and subsequently receives the immunogenic compositions, cells, and pharmaceutical formulations to treat remaining tumor cells. Additionally or alternatively, the subject can be administered the immunogenic compositions, cells, and pharmaceutical formulations before surgery or radiation. Such administration may be used to shrink the tumor prior to surgery.

### C. Individuals to be Treated

A subject in need of treatment is a subject having or at risk of having cancer or a subject having or at risk of having an infection (e.g., a subject having or at risk of contracting a viral that can lead to cancer). In some embodiments, the subject to be treated is a human subject at risk of developing a tumor caused by an infectious agent such as MCPyV, Hepatitis B virus (HBV), Hepatitis C virus (HCV), Human papilloma virus (HPV), Kaposi's sarcoma-associated herpesvirus, Epstein-Barr virus (EBV), or human T-cell lymphotropic virus. In some embodiments, the subject to be treated is a human subject with cancer caused by a viral infection. In one embodiment, the subject has Merkel cell carcinoma.

A subject having cancer is a subject that has detectable cancerous cells. “Cancer” as used herein refers to an uncontrolled growth of cells which interferes with the normal functioning of the bodily organs and systems. A subject at risk of developing a cancer is one who has a higher-than-normal probability of developing cancer. In preferred embodiments, a subject with a higher-than-normal probability of developing cancer is one who has a viral infection that can lead to cancer.

Examples of viruses that can be treated by the described methods, or for which the described methods confer protection, include, but are not limited to, Hepatitis B virus (HBV), Hepatitis C virus (HCV), Human papilloma virus (HPV), Kaposi's sarcoma-associated herpesvirus, Epstein-Barr virus (EBV), human T-cell lymphotropic virus. In some embodiments, the viruses that can be treated by the described methods, or for which the described methods confer protection are MCPyV. In some embodiments, the virally induced cancer that can be treated by the described methods, or for which the described methods confer protection is MCPyV-driven Merkel cell carcinoma.

### Cancer Caused by Viruses

The viruses that cause human cancer include hepatitis B virus (liver cancer), papillomaviruses (cervical and other anogenital cancers), Epstein-Barr virus (Burkitt's lymphoma and nasopharyngeal carcinoma), Kaposi's sarcoma-associated herpesvirus (Kaposi's sarcoma), and human T-cell lymphotropic virus (adult T-cell leukemia). In addition, hepatitis C virus (an RNA virus) is an indirect cause of liver cancers resulting from chronic tissue damage. In some embodiments, the compositions and methods thereof are used for preventing or treating an infection caused by one or more of these viruses, and/or cancer caused by one or more of these viruses. In such cases, the compositions include antigenic protein or isolated nucleic acid sequences encoding a viral protein and/or immunogenic domains and fragments thereof of the target virus. In preferred embodiments, the viral proteins include one or more from HPV-derived antigens (e.g., E2, E5, and E6), EBV-related antigens, hepatitis B or C Virus-related antigens, endogenous retrovirus (ERV)-derived antigens (including tumor-specific ERVs) and other tumor and infection-related antigens.

Given that the malignant transformation factors in MCC are viral antigens, a vaccine-based approach has been established to stimulate anti-tumor immunity and improve clinical outcomes. The methods are suited for treatment of MCC patients who are positive for Merkel Cell Polyomavirus (MCPyV).

In some embodiments, the subject to be treated is a human subject with an MCPyV infection. MCPyV infection can be combated or prevented by promoting in a patient a CD8⁺ immune response against cells infected with MCPyV. In some embodiments, cells infected with MCPyV express the Large T Antigen (LTA) protein of MCPyV or a fragment thereof. In other embodiments, cells infected with MCPyV express the LTA protein of MCPyV or a fragment thereof and have integrated within their genomes an MCPyV nucleotide sequence encoding the LTA protein of MCPyV or a fragment thereof.

MCPyV infection may be asymptomatic but can be tested and confirmed, e.g., by PCR amplification of viral DNA from a blood sample. In some embodiments, cells infected with MCPyV are in a tumoral state. In other embodiments, cells infected with MCPyV are not in a tumoral state. In asymptomatic patients, non-tumoral cells infected with MCPyV, in particular those expressing the LTA protein of MCPyV or a fragment thereof, are nevertheless indicative of a pre-tumoral status and it is therefore desirable to eliminate them because the patients are at risk of developing a tumor. Cells either expressing the LTA protein of MCPyV or a fragment thereof and/or having integrated within the genome an MCPyV nucleotide sequence encoding the LTA protein of MCPyV or a fragment thereof are indicative of a higher risk of developing a tumor.

Methods for inducing or stimulating an immune response against an MCPyV infection or MCPyV-driven Merkel cell carcinoma in the subject are described. The methods administer an effective amount of the disclosed formulations to a subject in need thereof, to elicit an immune response against an MCPyV infection or MCPyV-driven Merkel cell carcinoma, to prevent or treat one or more symptoms of an MCPyV infection or MCPyV-driven Merkel cell carcinoma. The methods are particularly suited for inducing or stimulating an immune response against MCPyV-driven Merkel cell carcinoma expressing one or more viral antigens such as the LTA of MCPyV.

Methods for inducing or stimulating a T cell mediated immune response against MCPyV or MCPyV-driven Merkel cell carcinoma in a human subject are also described. In preferred embodiments, the method is effective in inducing CD8⁺ T cells against MCPyV or MCPyV-driven Merkel cell carcinoma.

The compositions can be administered as an immunogenic composition or as part of vaccine, such as prophylactic vaccines, or therapeutic vaccines, which can be used to initiate or enhance a subject's immune response to a pre-existing antigen, such as a tumor antigen in a subject with cancer.

In some embodiments, prophylactic use cases for MCC are administered to selected populations, such as patients with lymphoproliferative disease associated with a higher risk of MCC (e.g. Chronic lymphocytic lymphoma) or patients who otherwise are or are going to be immunosuppressed (e.g. solid organ transplant patients taking immunosuppressive medications). Some methods optionally include a patient selection step.

The desired outcome of a prophylactic or therapeutic immune response may vary according to the disease, according to principles well known in the art. Similarly, immune responses against cancer, may alleviate symptoms, or may be one facet in an overall therapeutic intervention against a disease. For example, administration of the composition may reduce tumor size, or slow tumor growth compared to a control. The stimulation of an immune response against a cancer may be coupled with surgical, chemotherapeutic, radiologic, hormonal, and other immunologic approaches in order to affect treatment.

### D. Combination Therapies

The compositions can be further administered alone or in combination with one or more conventional therapies, or procedures for example, a conventional cancer therapy, radiation, or surgery.

In some embodiments, the conventional cancer therapy is in the form of one or more additional active agents. Therefore, in some embodiments, the compositions are administered in combination with one or more additional therapeutic agents. Conventional therapeutics agents are known in the art and can be determined by one of skill in the art based on the disease or disorder to be treated. For example, if the disease or condition is cancer, the compositions can be co-administered with a chemotherapeutic drug. The agents can be administered in the same or separate pharmaceutical composition from the antigen, adjuvant, or combination thereof.

The additional therapy or procedure can be simultaneous or sequential with the administration of the compositions. In some embodiments, the additional therapy is performed between drug cycles or during a drug holiday that is part of the composition dosage regime. For example, in some embodiments, the additional therapy or procedure is surgery, a radiation therapy, or chemotherapy.

Additional therapeutic agents include conventional cancer therapeutics such as chemotherapeutic agents, cytokines, chemokines, and radiation therapy, as discussed above. The majority of chemotherapeutic drugs can be divided into alkylating agents, antimetabolites, anthracyclines, plant alkaloids, topoisomerase inhibitors, and other antitumor agents. These drugs affect cell division or DNA synthesis and function in some way. Additional therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

In some embodiments, the additional therapy is a chemotherapeutic agent. Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal 66aunorubicin, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5 and combinations thereof.

In some embodiments, the compositions and methods are used prior to or in conjunction with an immunotherapy such as inhibition of checkpoint proteins such as components of the PD-1/PD-L1 axis or CD28-CTLA-4 axis using one or more immune checkpoint modulators (e.g., PD-1 antagonists, PD-1 ligand antagonists, LAG-3, and CTLA-4 antagonists), adoptive T cell therapy, and/or an additional cancer vaccine. Exemplary immune checkpoint modulators used in immunotherapy include Pembrolizumab (anti-PD1 mAb), Durvalumab (anti-PDL1 mAb), PDR001 (anti-PD1 mAb), Atezolizumab (anti-PDL1 mAb), Nivolumab (anti-PD1 mAb), Tremelimumab (anti-CTLA4 mAb), Avelumab (anti-PDL1 mAb), Relatimab (anti-LAG-3), APX005M (CD40 agonist mAb), and RG7876 (CD40 agonist mAb).

In some embodiments, the additional therapy is adoptive dendritic cell or T cell therapy. Methods of adoptive dendritic cell and T cell therapy are known in the art and used in clinical practice. Generally, adoptive T cell therapy involves the isolation and ex vivo expansion of tumor specific T cells to achieve greater number of T cells than what could be obtained by vaccination alone. The tumor specific T cells are then infused into patients with cancer in an attempt to give their immune system the ability to overwhelm remaining tumor via T cells, which can attack and kill the cancer. Several forms of adoptive T cell therapy can be used for cancer treatment including, but not limited to, culturing tumor infiltrating lymphocytes or TIL; isolating and expanding one particular T cell or clone; and using T cells that have been engineered to recognize and attack tumors. In some embodiments, the T cells are taken directly from the patient's blood. Methods of priming and activating T cells in vitro for adaptive T cell cancer therapy are known in the art. See, for example, Wang, et al, Blood, 109(11):4865-4872 (2007) and Hervas-Stubbs, et al, J. Immunol., 189(7):3299-310 (2012).

Historically, adoptive T cell therapy strategies have largely focused on the infusion of tumor antigen specific cytotoxic T cells (CTL) which can directly kill tumor cells. However, CD4+T helper (Th) cells such as Th1, Th2, Tfh, Treg, and Th17 can also be used. Th cells can activate antigen-specific effector cells and recruit cells of the innate immune system such as macrophages and dendritic cells to assist in antigen presentation (APC), and antigen primed Th cells can directly activate tumor antigen-specific CTL. As a result of activating APC, antigen specific Th₁ have been implicated as the initiators of epitope or determinant spreading which is a broadening of immunity to other antigens in the tumor. The ability to elicit epitope spreading broadens the immune response to many potential antigens in the tumor and can lead to more efficient tumor cell killing due to the ability to mount a heterogeneic response. In this way, adoptive T cell therapy can used to stimulate endogenous immunity.

In some embodiments, the T cells express an engineered TCR or chimeric antigen receptor (CARs, CAR T cells, or CARTs). Artificial T cell receptors are engineered receptors, which graft a particular specificity onto an immune effector cell. Typically, these receptors are used to graft the specificity of a monoclonal antibody onto a T cell and can be engineered to target virtually any tumor associated antigen. First generation CARs typically had the intracellular domain from the CD3 ζ-chain, which is the primary transmitter of signals from endogenous TCRs. Second generation CARs add intracellular signaling domains from various costimulatory protein receptors (e.g., CD28, 41BB, ICOS) to the cytoplasmic tail of the CAR to provide additional signals to the T cell, and third generation CARs combine multiple signaling domains, such as CD3z-CD28-41BB or CD3z-CD28-OX40, to further enhance effectiveness.

In some embodiments, the compositions and methods are used prior to or in conjunction with surgical removal of tumors, for example, in preventing primary tumor metastasis. In some embodiments, the compositions and methods are used to enhance body's own anti-tumor immune functions.

### E. Treatment Regimens

A treatment regimen can include one or multiple administrations of the compositions and formulations thereof for achieving a desired physiological change, including administering to an animal, such as a mammal, especially a human being, an effective amount of the compositions to treat the disease or symptom thereof, or to produce the physiological change. In preferred embodiments, the desired physiological change is to increase or activate or stimulate T cells specific against one or more viral proteins of a cancer-causing virus such as MCPyV or HPV. In some embodiments, the methods of treatment include administering to a subject, such as a mammal, especially a human being, an effective amount of the compositions to elicit increased immune responses against MCPyV-infected cells or MCPyV-driven Merkel cell carcinoma in the subject. In other embodiments, the methods of treatment include administering to a subject, such as a mammal, especially a human being, an effective amount of the compositions to elicit increased immune responses against HPV-infected cells or HPV-driven cancer cells in the subject.

### 1. Dosages and Effective Amounts

In some embodiments, methods include administering compositions include antigenic protein or a nucleotide sequence (e.g., mRNA) encoding one or more viral proteins of a cancer-causing virus such as MCPyV, or therapeutic T cell primed or engineered therewith, in an amount effective to treat the cancer. The methods typically administer to the subject an effective amount of the disclosed composition to increase the number of viral antigen-specific T cells, reduce cancer cell proliferation, reduce cancer cell metastasis, and/or reduce cancer cell viability in the subject.

Therapeutically effective amounts of the compositions used in the treatment of cancer are typically sufficient to reduce or alleviate one or more symptoms of cancer. Symptoms of cancer may be physical, such as tumor burden, or biological such as proliferation of cancer cells. Accordingly, the amount of the composition can be effective to, for example, kill tumor cells or inhibit proliferation or metastasis of the tumor cells. Preferably, the immunogenic compositions including antigenic protein or one or more nucleotide sequences (e.g., mRNA) encoding one or more viral proteins of a cancer-causing virus such as MCPyV, are preferentially delivered to target cells such as dendritic cells to assist in antigen presentation (APC). Preferably, the active agents do not target or otherwise modulate the activity or quantity of healthy cells not within or associated with tumor tissues, or do so at a reduced level compared to cancer or cancer-associated cells. In this way, by-products and other side effects associated with the compositions are reduced, preferably leading directly or indirectly to cancer cell death. In some embodiments, the compositions directly or indirectly reduce cancer cell migration, angiogenesis, immune escape, radioresistance, or a combination thereof. In some embodiments, the compositions directly or indirectly induce a change in the cancer cell itself or its microenvironment that suppresses proliferation of the cancer cells, or induces apoptosis of the cancer cells, or induces activation of an immune response against the cancer cells, or combinations thereof.

In some embodiments, the disclosed compositions are administered to a subject in a therapeutically effective amount to reduce tumor size. For example, in some embodiments, an effective amount of the compositions is used to put cancer in remission and/or keep the cancer in remission. Also provided are effective amounts of compositions to reduce or stop cancer stem cell proliferation.

The composition and/or the formulations thereof are administered to a subject in need thereof such as a human subject. In some embodiments, the subject had been primed, either by an infection with the virus or an immunization against the virus, either of which result in a balanced T cell and B cell immune response to the pathogen. In some embodiments, the dosage units include an effective amount for inducing or stimulating a protective T cell and/or B cell immune response to the virus.

The precise dosage will vary according to a variety of factors such as subject-dependent variables (e.g., age, immune system health, clinical symptoms etc.). Persons of ordinary skill can determine optimum dosages, dosing methodologies and repetition rates, which may vary depending on the relative potency of individual vaccines, and can generally be estimated based on effective dosages (e.g., an EC50 measured by antigen specific T cells) in ex vivo assay and in in vivo animal models.

The disclosed compositions and formulations produce prophylactically- and/or therapeutically efficacious levels, concentrations and/or titers of antigen-specific antibodies and/or antigen-specific T cells in the blood or serum of a subject to whom it is administered.

In some embodiments, a single deposition of the composition or immunogenic formulations thereof is required to elicit a long-lasting, potent antigen-specific immune response in the subject. In some embodiments, the disclosed compositions are administered on a dosage schedule, for example, an initial administration of a vaccine with subsequent booster administrations. Thus, in some embodiments, the composition is administered 2, 3, 4, or more times, for example, 1, 2, 3, 4, 5, 6, 7, 8, 9, 10, 11, 12, 13, 14, or 15 days, weeks, months, or years apart. Dosage regimens or cycles of the compositions and/or additional agents can be completely or partially overlapping or can be sequential. In some embodiments, a second dose of the vaccine is administered anywhere from two weeks to one year, from one to six months, after the initial administration. Additionally, a third dose may be administered after the second dose and from three months to two years, or even longer, 4 to 6 months, or 6 months to one year after the initial administration. The boosting antigen may be administered using the same composition, or as a whole protein, an immunogenic peptide fraction of the protein, DNA or RNA encoding the protein or peptide. In some embodiments, no booster immunization is required.

### 2. Controls

The therapeutic result of the compositions activity can be compared to a control. Suitable controls are known in the art and include, for example, untreated cells or an untreated subject. A typical control is a comparison of a condition or symptom of a subject prior to and after administration of the compositions. The condition or symptom can be a biochemical, molecular, physiological, or pathological readout. For example, the effect of the composition on a particular symptom, pharmacologic, or physiologic indicator can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known in the art.

### VII. Kits

Kits are also disclosed. The kit can include a single dose or a plurality of doses of a composition including isolated nucleic acid sequences (e.g., a viral vector, mRNA) encoding one or more viral antigens of one or more viruses that cause cancer, and/or immunogenic domains and fragments thereof, or pharmaceutical formulation thereof, and instructions for administering the compositions. Specifically, the instructions direct that an effective amount of the composition be administered to an individual at risk of exposing a cancer-causing virus or developing cancer caused by the virus.

Dosage units of the composition alone or in combination with adjuvant in a pharmaceutically acceptable carrier for shipping and storage and/or administration are also provided, and can form part of a kit, for example a vaccination kit.

The compositions can be packaged in single or multi-vial kits that contain all of the components needed to prepare the complexes. A multi-vial kit preferably contains the same general components but employs more than one vial in reconstituting the compositions. In some embodiments, the contents of one or more vials are lyophilized and/or required to be frozen. Thus, in some embodiments, the contents of one or more vials are reconstituted and/or thawed prior to administration to a subject.

Kits can also contain syringes of various capacities or vessels with deformable sides (e.g., plastic vessels or plastic-sided vessels) that can be squeezed to force a liquid composition out of an orifice. The size and design of the syringe will depend on the route of administration. For example, in one embodiment, a syringe for administering the compositions locally, may be capable of accurately delivering a smaller volume. Larger syringes, pumps and/or catheters can also be provided. Any of the kits can include instructions for use.

The invention can be further understood by the following numbered paragraphs:
1. An immunogenic composition including a nucleic acid encoding a viral antigen or an immunogenic fragment thereof, and optionally an adjuvant, wherein the viral antigen is derived from a virus that causes cancer.
2. The immunogenic composition of paragraph 1, wherein the viral antigen is derived from a virus selected from the group consisting of Merkel Cell Polyomavirus (MCPyV), Hepatitis B virus (HBV), Hepatitis C virus (HCV), Human papilloma virus (HPV), Kaposi's sarcoma-associated herpesvirus, Epstein-Barr virus (EBV), and human T-cell lymphotropic virus.
3. The immunogenic composition of paragraph 1 or 2, wherein the viral antigen is derived from MCPyV or HPV.
4. The immunogenic composition of any one of paragraphs 1-3, wherein the viral antigen is derived from a truncated form of the viral Large T Antigen (LTA) or Small T Antigen (STA) of MCPyV, or derived from E2, E5, or E6 proteins of HPV.
5. The immunogenic composition of any one of paragraphs 1-4, wherein the nucleic acid further includes a nucleotide sequence encoding a signal peptide.
6. The immunogenic composition of paragraph 5, wherein the signal peptide is derived from a signal peptide of a full-length coronavirus spike protein.
7. The immunogenic composition of paragraph 6, wherein the signal peptide is derived from a full-length coronavirus spike protein of a coronavirus variant of SARS-CoV-2 selected from the group consisting of SARS-CoV-2 B.1.1.7 (Alpha variant), SARS-CoV-2 B.1.351 (Beta variant), SARS-CoV-2 P.1 (Gamma variant), SARS-CoV-2 B.1.617, SARS-CoV-2 B.1.617.1 (Kappa variant), SARS-CoV-2 B.1.621 (Mu variant), SARS-CoV-2 B.1.617.2 (Delta variant), SARS-CoV-2 B.1.617.3, and SARS-CoV-2 B.1.1.529 (Omicron variant).
8. The immunogenic composition of paragraph 6 or 7, wherein the signal peptide includes the nucleotide sequence of SEQ ID NO:11.
9. The immunogenic composition of any one of paragraphs 1-8, the nucleic acid further includes a nucleotide sequence encoding an affinity tag.
10. The immunogenic composition of paragraph 9, wherein the affinity tag is FLAG-tag having the amino acid sequence DYKDDDDK (SEQ ID NO: 21).
11. The immunogenic composition of any one of paragraphs 6-10, wherein the nucleic acid includes a nucleotide sequence encoding a viral antigen or an immunogenic fragment thereof, a signal peptide at the N-terminus, and an affinity tag at the C-terminus of the viral antigen or an immunogenic fragment thereof.
12. The immunogenic composition of any one of paragraphs 1-11, wherein the nucleic acid further includes a 5′ untranslated region (UTR) sequence.
13. The immunogenic composition of paragraph 12, wherein the 5′ UTR sequence includes the nucleotide sequence of any one of SEQ ID NOs:16-19.
14. The immunogenic composition of any one of paragraphs 1-13, wherein the nucleic acid further includes a 3′ UTR sequence.
15. The immunogenic composition of paragraph 14, wherein the 3′ UTR sequence includes the nucleotide sequence of SEQ ID NO:20.
16. The immunogenic composition of any one of paragraphs 1-15, wherein the nucleic acid further includes a poly(A) tail.
17. The immunogenic composition of any one of paragraphs 1-16, wherein the viral antigen or an immunogenic fragment thereof includes the amino acid sequence of any one of SEQ ID NOs:1-5 or 25, and a variant thereof having at least 70%, 75%, 80%, 85%, 90%, 91%, 92%, 93%, 94%, 95%, 96%, 97%, 98%, or 99% sequence identity thereto.
18. The immunogenic composition of any one of paragraphs 1-16, wherein the nucleic acid includes the nucleotide sequence of any one of SEQ ID NOs:6-10 or 24.
19. The immunogenic composition of any one of paragraphs 1-18, wherein the nucleic acid is an mRNA.
20. A double stranded DNA sequence including the immunogenic composition of any one of paragraphs 1-18.
21. The double stranded DNA sequence of paragraph 20 further including (i) one or more restriction sites, (iii) promoter region, (iv) TRILINK CAP site, and/or (v) traditional KOZAK sequence.
22. The double stranded DNA sequence of paragraph 21, wherein the promoter region is a T7 promoter, a T3 promoter, or a SP6 promoter.
23. The double stranded DNA sequence of any one of paragraphs 20-22 includes the nucleotide sequence of any one of SEQ ID NOs:6-10, or 24.
24. A pharmaceutical formulation including the immunogenic composition of any one of paragraphs 1-19, and one or more pharmaceutically acceptable carrier.
25. The pharmaceutical formulation of paragraph 24, wherein the immunogenic composition is an mRNA.
26. The pharmaceutical formulation of paragraph 24 or 25, wherein the immunogenic composition is encapsulated within and/or associated with a delivery vehicle that increases the serum half-life of the immunogenic composition as compared to the serum half-life of the same amount of the immunogenic composition alone.
27. The pharmaceutical formulation of paragraph 26, wherein the delivery vehicle is a lipid nanoparticle.
28. The pharmaceutical formulation of paragraph 27, wherein the lipid nanoparticle is formulated with SM-102, 1,2-DSPC, cholesterol, and DMG-PEG.
29. The pharmaceutical formulation of paragraph 27 or 28, wherein the lipid nanoparticle is formulated with SM-102, 1,2-DSPC, cholesterol, and DMG-PEG in a lipid molar ratio of 50:10:38.5:1.5.
30. The pharmaceutical formulation of any one of paragraphs 24-29 formulated for intranasal or by intramuscular administration.
31. A method of eliciting an immune response in a subject in need thereof, including administering to the subject an effective amount of the pharmaceutical formulation of any one of paragraphs 24-30.
32. The method of paragraph 31, wherein the pharmaceutical formulation is administered by intranasally or by intravascular or intramuscular injection.
33. The method of paragraph 31 or 32, wherein the subject has or is at risk of having a cancer-associated viral infection.
34. The method of any one of paragraphs 31-33, wherein the subject has or is at risk of having a viral infection caused by one or more of Merkel Cell Polyomavirus (MCPyV), Hepatitis B virus (HBV), Hepatitis C virus (HCV), Human papilloma virus (HPV), Kaposi's sarcoma-associated herpesvirus, Epstein-Barr virus (EBV), and human T-cell lymphotropic virus.
35. The method of any one of paragraphs 31-34, wherein the pharmaceutical formulation is administered to the subject having or at risk of developing cancer.
36. The method of any one of paragraphs 31-35, wherein the subject has a viral infection that can lead to virally driven cancer or the virally driven cancer.
37. The method of paragraph 36, wherein the cancer is selected from the group consisting of Merkel cell carcinoma, liver cancer, cervical and other anogenital cancers, Burkitt's lymphoma, nasopharyngeal carcinoma, Kaposi's sarcoma, and adult T-cell leukemia.
38. The method of paragraph 36 or 37, wherein the cancer is Merkel cell carcinoma.
39. A method for enriching T cells specific for a viral or tumor antigen, including
optionally i) isolating T cells from a subject; and
ii) stimulating the T cells using the viral or tumor antigen or a nucleic acid encoding the same, optionally wherein the viral or tumor antigen is encoded by the nucleic acid of any one of paragraphs 1-23.
40. The method of paragraph 39, further including one or more culturing steps before and/or after the step ii) of stimulating the T cells using the viral or tumor antigen.
41. The method of paragraph 40, wherein the culturing step is carried out in the presence of one or more cytokines selected from the group consisting of IL-2, IL-7, and IL-15.
42. The method of any one of paragraphs 39-41, wherein the viral or tumor antigen for stimulating the T cells are expressed and presented by dendritic cells, optionally monocyte-derived dendritic cells.
43. The method of paragraph 42, wherein the dendritic cells are derived from the same or different subject as the T cells.
44. The method of any one of paragraphs 39-43, wherein the dendritic and/or T cells are isolated from peripheral blood mononuclear cells of the subject.
45. The method of any one of paragraphs 39-44, wherein the subject is one with a viral infection that can lead to cancer or a virally driven cancer.
46. The method of any one of paragraphs 39-45, wherein the subject has MCC.
47. The method of any one of paragraphs 39-46, further including the step of isolating T cells that are specific towards the viral or tumor antigen.
48. The method of any one of paragraphs 39-47, further including the step of sequencing the T cell receptors (TCRs) from the enriched T cells.
49. The method of paragraph 48, further including the step of determining the binding characteristics of the TCRs towards the viral or tumor antigen.
50. The method of paragraph 49, further including the step of selecting TCRs based on desired binding characteristics.
51. The method of paragraph 50, further including the step of using one or more of these TCRs with desired binding characteristics to engineer T cells expressing the TCR, and optionally using the engineered cells in T cell therapy. 52. The method of paragraph 51, wherein the T cell therapy is adoptive transfer of one or more T cells expressing one or more of these TCRs with desired binding characteristics.
53. An engineered T cell receptor (TCR) including an alpha chain variable domain including the amino acid sequence of SEQ ID NO:27, 29, 31, 33, 35, 37, 39, 41, 43, or 45, or a variant thereof with at least 70% sequence identity thereto, and/or a beta chain variable domain including the amino acid sequence of SEQ ID NO:28, 30, 32, 34, 36, 38, 40, 42, 44, or 46, or a variant thereof with at least 70% sequence identity thereto, wherein the TCR is specific for an LTA antigen.
54. The TCR of paragraph 53, wherein the engineered TCR includes
an alpha chain variable domain including the amino acid sequence of SEQ ID NO:27 and a beta chain variable domain including the amino acid sequence of SEQ ID NO:28;
an alpha chain variable domain including the amino acid sequence of SEQ ID NO:29 and a beta chain variable domain including the amino acid sequence of SEQ ID NO:30;
an alpha chain variable domain including the amino acid sequence of SEQ ID NO:31 and a beta chain variable domain including the amino acid sequence of SEQ ID NO:32;
an alpha chain variable domain including the amino acid sequence of SEQ ID NO:33 and a beta chain variable domain including the amino acid sequence of SEQ ID NO:34;
an alpha chain variable domain including the amino acid sequence of SEQ ID NO:35 and a beta chain variable domain including the amino acid sequence of SEQ ID NO:36;
an alpha chain variable domain including the amino acid sequence of SEQ ID NO:37 and a beta chain variable domain including the amino acid sequence of SEQ ID NO:38;
an alpha chain variable domain including the amino acid sequence of SEQ ID NO:39 and a beta chain variable domain including the amino acid sequence of SEQ ID NO:40;
an alpha chain variable domain including the amino acid sequence of SEQ ID NO:41 and a beta chain variable domain including the amino acid sequence of SEQ ID NO:42;
an alpha chain variable domain including the amino acid sequence of SEQ ID NO:43 and a beta chain variable domain including the amino acid sequence of SEQ ID NO:44; or
an alpha chain variable domain including the amino acid sequence of SEQ ID NO:45 and a beta chain variable domain including the amino acid sequence of SEQ ID NO:46.
55. The TCR of paragraphs 53 or 54, wherein the alpha and beta variable domains each include three complementarity determination regions: CDR1, CDR2, and CDR3.
56. The TCR of any one of paragraphs 53-55, wherein the CDR3 of the alpha variable domain is SEQ ID NO:27, 29, 31, 33, 35, 37, 39, 41, 43, or 45, or a variant thereof with at least 70% sequence identity thereto and the CDR3 of the beta variable domain is SEQ ID NO:28, 30, 32, 34, 36, 38, 40, 42, 44, or 46, or a variant thereof with at least 70% sequence identity thereto.
57. The TCR of any one of paragraphs 53-56, wherein the TCR is humanized.
58. The TCR of any one of paragraphs 53-57, wherein the TCR is further defined as a soluble TCR, wherein the soluble TCR does not include a transmembrane domain, or includes transmembrane domain that is a CD28 transmembrane domain or a CD8a transmembrane domain, or further includes a T-cell signaling domain of any one of the following proteins: a human CD8-alpha protein, a human CD28 protein, a human CD3-zeta protein, a human FcRγ protein, a CD27 protein, an OX40 protein, a human 4-1BB protein, or any combination of the foregoing.
59. The TCR of any one of paragraphs 53-58, the TCR further including a detectable label.
60. The TCR of any one of paragraphs 53-59, wherein the TCR is covalently bound to a therapeutic agent, an immunotoxin or a chemotherapeutic agent.
61. A polypeptide encoding the TCR of paragraphs 60.
62. A nucleic acid encoding the polypeptide of any one of paragraphs 53-61.
63. An expression vector encoding the TCR of any one of paragraphs 53-62.
64. The expression vector of paragraph 63, wherein the sequence encoding the TCR is under the control of a promoter.
65. The expression vector of paragraphs 63 or 64, wherein the expression vector is a viral or a retroviral vector.
66. The expression vector of any one of paragraphs 63-65, wherein the vector further encodes a linker domain positioned between the alpha chain and beta chain.
67. The expression vector of paragraph 66, wherein the linker domain includes one or more protease cleavage sites, or wherein the one or more cleavage sites are separated by a spacer.
68. A host cell engineered to express the TCR of any one of paragraphs 53-60.
69. The host cell of paragraph 68, wherein the cell is a T cell.
70. A method of adoptive T cell therapy including administering a subject in need thereof an effective amount of the T cells (i) primed or engineered according to any the methods of paragraphs 39-52, or (ii) of paragraph 69.
71. A method of maturing dendritic cells (DC) optionally Mo-DC, including
optionally i) isolating DC from a subject; and
ii) stimulating the DC using the viral or tumor antigen or a nucleic acid encoding the same, optionally wherein the viral or tumor antigen is encoded by the nucleic acid of any one of paragraphs 1-23.
72. A method of adoptive dendritic cell therapy including administering a subject in need thereof dendritic cells matured according to the method of paragraph 71.
73. The method of any one of paragraphs 70-72, wherein the subject has or is at risk of developing MCC.

The present invention will be further understood by reference to the following non-limiting examples.

### EXAMPLES

### Example 1: LTA Plasmid Design, B16 Mouse Melanoma LTA+ and Empty Vector Control Creation

There is no murine model of MCC and no analogous cancer that expresses the MCPyV LTA; therefore, B16 mouse melanoma cells were engineered to express the MCPyV LTA to serve as a model. A consensus sequence for the truncated LTA was identified by comparison with sequenced human MCC tumors and adjusted based on the known protein functional domains (Cheng J, et al., J Virol. 2013 June; 87(11):6118-26). Restriction sites NheI and MluI (shown below in bold) were added to flank the sequence, and a traditional Kozak sequence was added prior to the start codon. A FLAG sequence was added to one set of plasmids so that the protein could be identified by two separate markers; this sequence was designated LTAF.

(SEQ ID NO: 26)
GAGCTCGCTAGCGCCACCatggatttagtcctaaataggaaagaaagagag
gctctctgcaagcttttagagattgctcctaattgttatggcaacatccct
ctgatgaaagctgctttcaaaagaagctgcttaaagcatcaccctgataaa
gggggaaatcctgttataatgatggaattgaacaccctttggagcaaattc
cagcaaaatatccacaagctcagaagtgacttctctatgtttgatgaggtc
gacgaggcccctatatatgggaccactaaattcaaagaatggtggagatca
ggaggattcagcttcgggaaggcatacgaatatgggcccaatccacacggg
accaactcaagatccagaaagccttcctccaatgcatccaggggagccccc
agtggaagctcaccaccccacagccagagctcttcctctgggtatgggtcc
ttctcagcgtcccaggcttcagactcccagtccagaggacccgatatacct
cccgaacaccatgaggaacccacctcatcctctggatccagtagcagagag
gagaccaccaattcaggaagagaatccagcacacccaatggaaccagtgta
cctagaaattcttccagaacggatggcacctgggaggatctcttctgcgat
gaatcactttcctcccctgagcctccctcgtcctctgaggagcctgaggag
cccccctcctcaagaagctcgccccggcagcccccgtcttcctctgccgag
gaggcctcgtcatctcagtttacagattagACGCGTGAGCTC.

The bolded sequences identify restriction sites for cloning, the sequence in italics is a Kozak sequence, and the lowercase sequence is the nucleotide sequence that encodes the polypeptide.

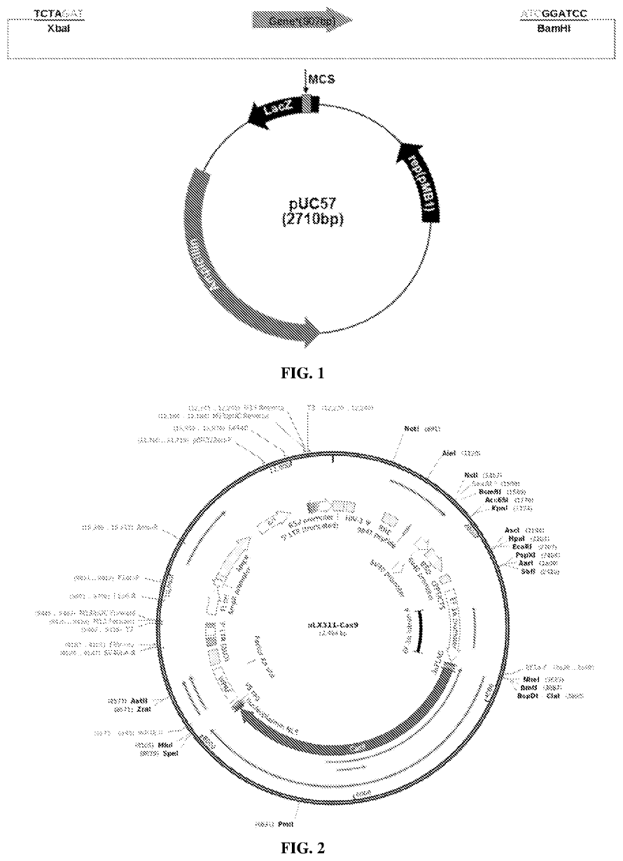

The above sequence was then synthesized into a pUC57 plasmid with ampicillin resistance by GenScript (Piscataway, NJ; FIG. 1). Commercial plasmids were then digested with NheI and MluI restriction enzymes to extract the template LTA sequence. A pLX311 vector plasmid with blasticidin resistance was selected for transduction. The same restriction sites in pLX311 were used to cut out Cas9 and place the template sequence in front of the EF1a Promoter, as shown below (FIG. 2).

A subset of the pLX311 plasmids only had the Cas9 region removed, and no coding sequence inserted. These were designated the empty vector (EV) control. All plasmids were expanded in Top10 E. Coli using a Qiagen (Hilden, Germany) MiniPrep kit. Plasmids for LTA and EV were tested by transfection into HEK 293T cells using Polyplus (Illkirch, France) JetOptimus and confirmed by western blot to express the appropriate proteins using both LTA directed antibodies (Santa Cruz Biotechnology, Dallas, TX) and FLAG tag antibodies (Sigma, St. Louis, MO).

Once expression was confirmed, Lentivirus containing the LTA and EV plasmids in psPAX2 vectors was produced in 293T cells. B16 mouse melanoma cells were then infected with the lentiviral vectors for transduction of the LTA or EV plasmids. Infected cells underwent blasticidin selection to confirm transduction. Expression of LTA protein in the selected cells was confirmed by western blot as described above, and cell lines with high expression were chosen for expansion and tumor engraftment (data now shown).

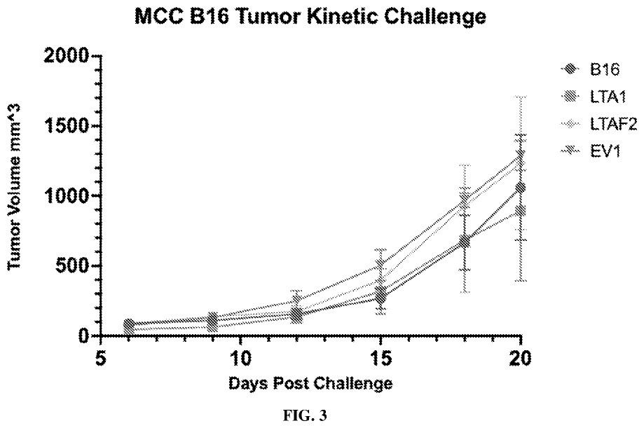

In order to establish that there was no difference in growth kinetics between LTA expressing tumors and control tumors, C57BL/6J mice were challenged with the different cell lines and tumor volume was tracked every 3 days for 20 days. Each mouse received 1 million cells suspended in 200 μL of HBSS injected subcutaneously into the right flank on day zero (FIG. 3).

There was no significant difference in growth rate between the four tumor types. The EV1 and LTAF2 lines were selected for challenge and treatment with the LTA mRNA vaccine.

### Example 2: MRNA Template Plasmid Design

Using the consensus sequence for the truncated MCPyV LTA protein as a template, necessary components were added for a functional mRNA subunit. A T7 promoter sequence was added immediately following the first restriction site for compatibility with commercial in-vitro transcription kits. This T7 promoter sequence was modified to end with an AGG sequence for compatibility with Trilink (San Diego, CA) CleanCap 5′ mRNA capping technology. For the 5′ untranslated region (UTR), a synthetic sequence was chosen based on previously published screen in Nature Communications by Cao et al in 2021 (Cao, J et al., Nat Commun 12, 4138 (2021)). This sequence was generated by artificial intelligence to be optimized for high efficiency translation and was among the highest performers in their 5′ UTR screen. Constructs with and without a signal peptide sequence from the SARS-CoV-2 spike protein after the start codon were generated with the hypothesis that this sequence could theoretically assist with trafficking of the mRNA for efficient translation. This signal peptide was chosen because of its previously demonstrated success in multiple COVID19 mRNA-based vaccine strategies as it otherwise does not exist in the MCPyV genome. The natural 3′ UTR from the Human HBA1 gene was selected because of its demonstrated success in prior mRNA constructs and short length, which is predicted to minimize secondary structure formation and facilitate translation while preventing or delaying degradation. A 100 base polyadenylation sequence was added following the 3′UTR.

### LTA Vaccine Sequence:

(SEQ ID NO: 24)
GAGCTCACGCGTTAATACGACTCACTATAAGACCCAAGCTGGCTAGCGTTT
AAACTTAAGCTTGGTACCGCTTGTCTCGCTCCGGGGAACGCTCGGAAACTC
CCGGCCGCCGCCACCCGCGTCTGTTCTGTTACACAAGGGAAGAAAAGCCGC
TGCCGCACTCCGAGTGTGCCACCAtgTTCGTGTTCCTGGTGCTGCTGCCCC
TGGTGAGCAGCCAGTGCGTGgatttagtcctaaataggaaagaaagagagg
ctctctgcaagcttttagagattgctcctaattgttatggcaacatccctc
tgatgaaagctgctttcaaaagaagctgcttaaagcatcaccctgataaag
ggggaaatcctgttataatgatggaattgaacaccctttggagcaaattcc
agcaaaatatccacaagctcagaagtgacttctctatgtttgatgaggtcg
acgaggcccctatatatgggaccactaaattcaaagaatggtggagatcag
gaggattcagcttcgggaaggcatacgaatatgggcccaatccacacggga
ccaactcaagatccagaaagccttcctccaatgcatccaggggagccccca
gtggaagctcaccaccccacagccagagctcttcctctgggtatgggtcct
tctcagcgtcccaggcttcagactcccagtccagaggacccgatatacctc
ccgaacaccatgaggaacccacctcatcctctggatccagtagcagagagg
agaccaccaattcaggaagagaatccagcacacccaatggaaccagtgtac
ctagaaattcttccagaacggatggcacctgggaggatctcttctgcgatg
aatcactttcctcccctgagcctccctcgtcctctgaggagcctgaggagc
ccccctcctcaagaagctcgccccggcagcccccgtcttcctctgccgagg
aggcctcgtcatctcagtttacagattagGCTGGAGCCTCGGTGGCCTAGC
TTCTTGCCCCTTGGGCCTCCCCCCAGCCCCTCCTCCCCTTCCTGCACCCGT
ACCCCCGTGGTCTTTGAATAAAGTCTGAGTGGGCGGCAAAAAAAAAAAAAA
AAAAAAAAAAAAAAAAAAAAAAAAAAAAAAAAAAAAAAAAAAAAAAAAAAA
AAAAAAAAAAAAAAAAAAAAAAAAAAAAAAAAAAAATCTAGAGAGCTC

This sequence was then synthesized into a pUC57-Simple plasmid with an ampicillin resistance gene by Genscript. The mRNA template sequence is shown in purple between MluI and XbaI restriction sites (FIG. 4).

### Example 3: MRNA Production and Lipid Nanoparticle Assembly

### Material and Methods

The template DNA was extracted from the base plasmids by cutting at restriction sites MluI and XbaI. Template DNA was separated from plasmid DNA based on fragment size by gel electrophoresis and was purified from agarose gel using a Zymo Research (Irvine, CA) Zymoclean Gel DNA recovery kit. Purified, double stranded, linearized template DNA was then used for mRNA production via the New England Biolabs (Ipswich, MA) HiScribe T7 High Yield RNA Kit with addition of Trilink CleanCap mRNA capping technology and Trilink N1-MethylPseudo-UTP modified Uracil base for increased stability and translational efficiency of the mRNA product. The mRNA product was then purified using the New England Biolabs Monarch RNA Cleanup Kit. Purified functional mRNA was then stored in sterile water at −80 degrees.

For injection into animals, lipid nanoparticles containing commercially available SM-102, 1,2-DSPC, cholesterol, and DMG-PEG in a lipid molar ratio of 50:10:38.5:1.5 were assembled and incorporated with mRNA using a rapid solvent injection mixing technique. mRNA was first combined with 50 mM sodium acetate solution at pH 5.0. This solution was then stirred in a sterile container at 700 rpm and the ethanolic mixture was rapidly injected into the acidic solution and mixed for an additional 30 minutes to create homogenous nanoparticles containing the functional mRNA units. Placebo containing all components except for mRNA were prepared using the same process. The nanoparticles were then purified by dialysis against sterile phosphate buffered saline using 3.5 k MWCO dialysis cassettes (Thermo). The nanoparticle solution was then drawn into sterile 1 mL syringes and stored at 4 degrees until use.

### Results

The functionality of the mRNA product was tested by in-vitro transfection into A375, 293T, and B16 cells using Lipofectamine 2000 (Thermo Fisher Scientific, Waltham, MA). Protein production was confirmed by western blot using MCPyV LTA antibodies as described above. Only signal peptide-containing constructs (SP) were detectably expressed following transfection, indicating that this extra engineering step is required for efficient cellular production of the vaccine antigen.

### Example 4: Modified B16 Tumor Engraftment and LTA Vaccine Treatment

### Material and Methods

Groups of 5 female C57BL/6J mice were challenged with 1 million cells of either LTAF or EV modified B16 lines in HBSS by subcutaneous injection into the right flank on day zero. Tumor volume was tracked every 3 days along with survival. Endpoints included tumor volume greater than 2000 mm³, greater than 25% tumor ulceration, ulceration with visible blood, and severe lethargy or dehydration. Mice were treated with 6 ug or 15 ug of vaccine or volume matched placebo by a single intramuscular injection into the left flank (contralateral to tumor) on day 6 (6 ug) or day 9 (15 ug).

### Results

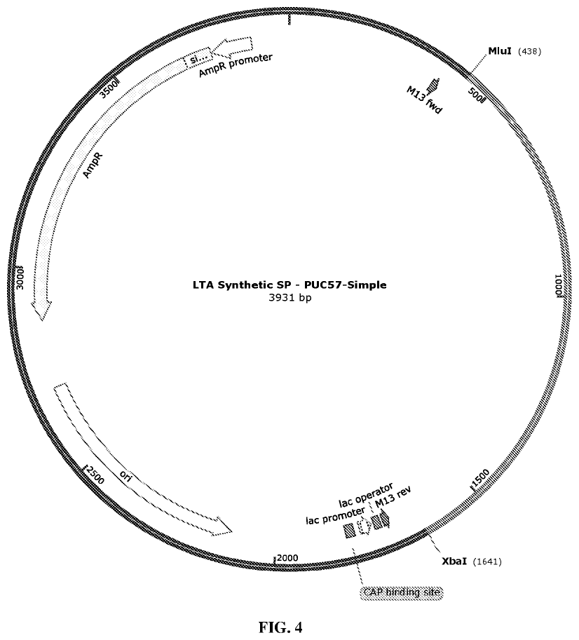

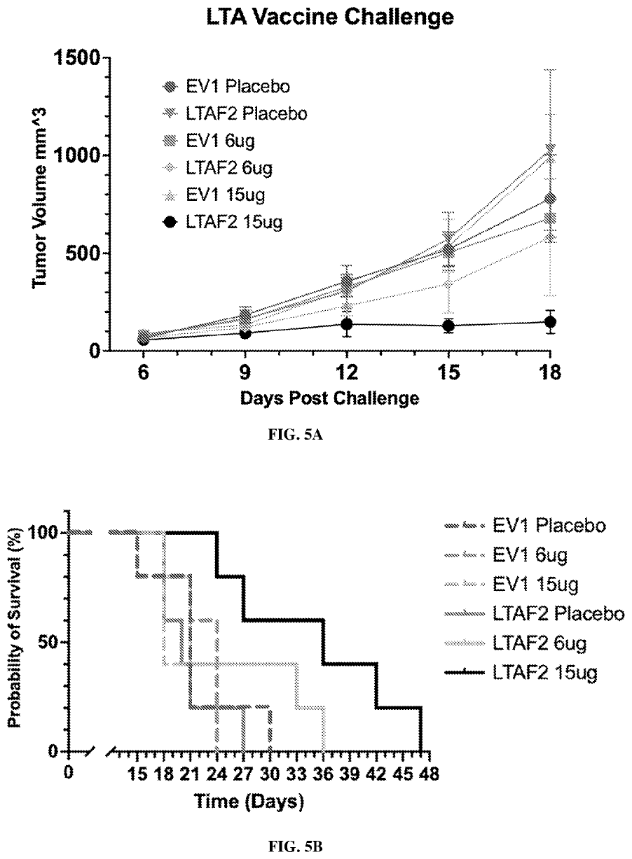

Tumor growth was significantly suppressed, and survival was significantly increased by 15 ug vaccination in the LTAF tumor group compared to both EV+vaccine and LTAF+placebo (FIGS. 5A-5B). The 6-μg treatment group experienced a non-statistically significant initial suppression in growth and increase in survival compared to placebo groups. There was mild erythema at the intramuscular injection site, but no other adverse reactions to vaccination were noted. Specifically, there was no indication of secondary infection following vaccination. The placebo groups also experienced mild erythema at the intramuscular injection site, but no other adverse reactions to placebo were noted.

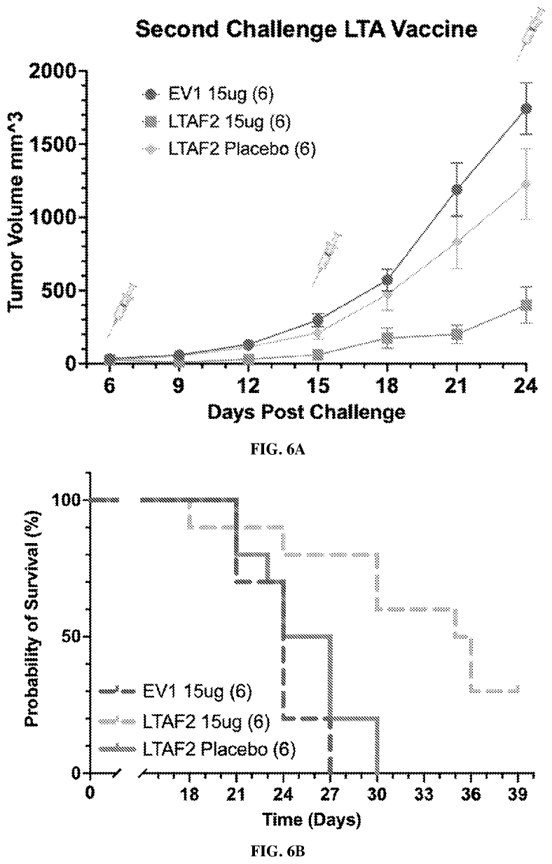

Based on the results of the above challenge, additional groups of 10 female C57BL/6J mice were challenged with 1 million cells of either LTAF or EV modified B16 lines in HBSS by subcutaneous injection into the right flank on day zero. Tumor volume and survival were again tracked using the same endpoints. Mice were treated with 15 ug of LTA vaccine or volume matched placebo on day 6, 15, and 24 (FIGS. 6A-6B).

Treatment with 15 ug of LTA mRNA vaccine on day 6, 15, and 24 significantly suppressed tumor growth and increased survival in the LTAF tumor group compared to both EV+vaccine and LTAF+placebo. Other than erythema at the intramuscular injection site, no adverse reactions to vaccination or placebo were noted.

### Example 5: Validation Using MCC Patient Samples

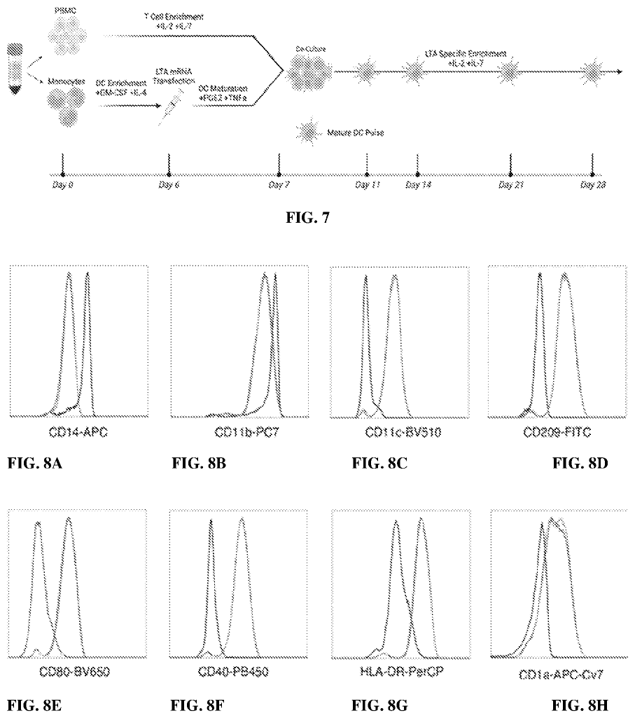

An approach was developed to expand vaccine-specific T cells from peripheral blood or another source of immune cells (e.g., leukopaks). Briefly, monocyte-derived dendritic cells (DCs) are isolated using magnetic columns and cultured in GM-CSF and IL-4. T cells are enriched using IL-2 and IL-7. The mRNA vaccine is introduced into monocyte-derived DCs using transfection as in the experiments that immediately follow, electroporation or another method and the DCs are used to stimulate the T cells repeatedly, enriching for vaccine-specific T cell clonotypes (FIG. 7).

Specifically, following isolation of patient PBMC, monocytes were isolated using a CD14+ magnetic selection kit and enriched monocyte-derived dendritic cells (Mo-DC) from a patient with MCC known to be associated with MCPyV using GM-CSF and IL-4. Mo-DC were incubated with either lipofectamine alone (Mo-DC Placebo) or lipofectamine with LTA mRNA vaccine. Mo-DC Placebo and Mo-DC LTA were incubated with PGE2 and TNF in order to induce maturation and enhance their antigen presentation capabilities (FIGS. 8A-8H).

Using western blotting, it was confirmed that the vaccine candidate was expressed well in matured Mo-DC following transfection.

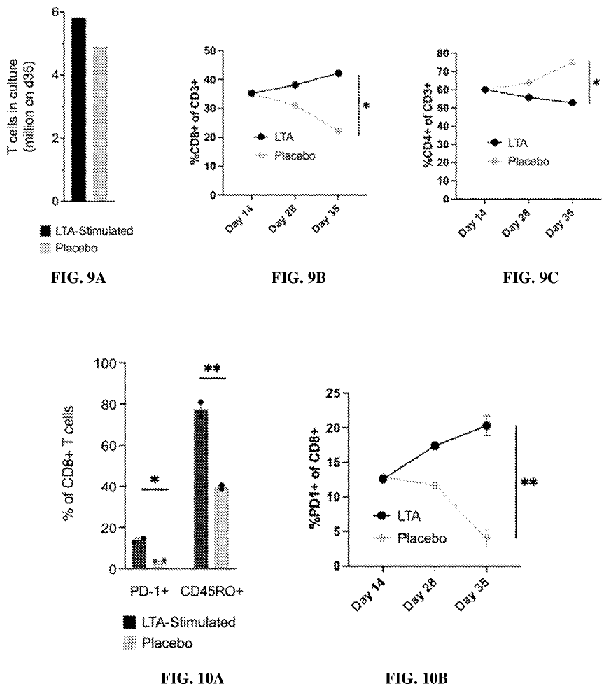

During DC enrichment, maturation and transfection, the remainder of patient PBMC were incubated in IL-2 and IL-7 for one week to enrich cultures for T cells. T cell cultures were stimulated with either Placebo Mo-DCs or LTA Mo-DCs with the goal of enriching LTA-specific T cells following the schedule outlined in FIG. 7. On day 35 of culture following 5 DC pulses, enrichment was observed in T cells and CD8+ T cells as a fraction of cells in culture (FIGS. 9A-9C).

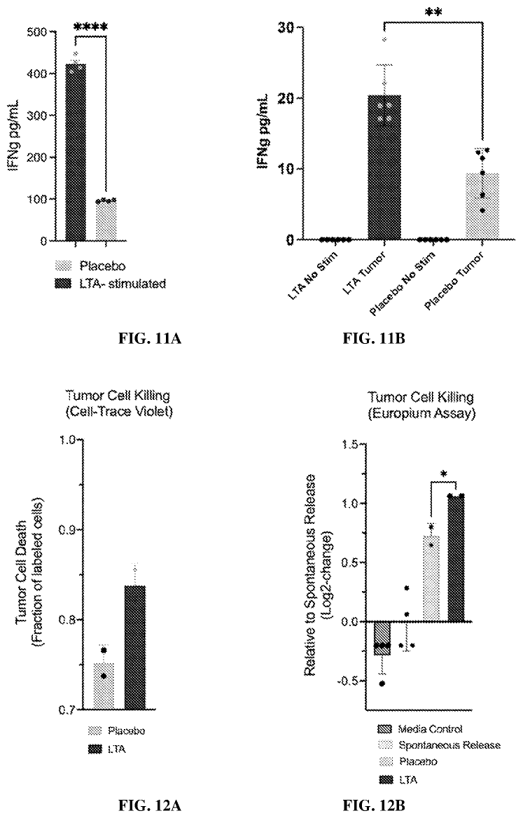

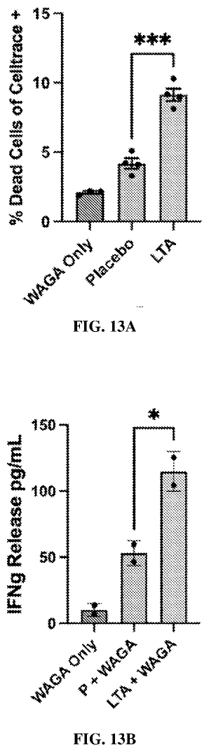

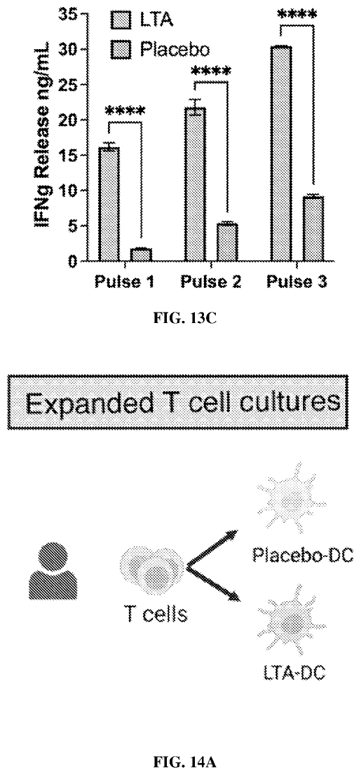

In addition to enrichment of the fraction of CD8+ T cells in culture, increases were observed in the expression of PD-1 and in the expression of CD45RO on the surface of CD8+ T cells, indicating activation and the generation of memory phenotypes, respectively (FIGS. 10A-10B).

On day 28 of culture following repeated stimulation with Placebo vs LTA vaccine Mo-DC, T cells were stimulated as per planned protocol. To assess T cell response to LTA or placebo stimulation, supernatants were collected after 48 hours, and IFN-gamma ELISA was performed (FIG. 11A). This assay demonstrated enhanced T cell response to stimulation by T cells stimulated with LTA Mo-DC compared to Placebo Mo-DCs, indicating that the vaccine induced LTA-specific T cell function as well as the enrichment of activated CD8+ T cells. Furthermore, when LTA enriched T cells and placebo T cells were exposed to patient-matched MCC tumor cells, the LTA enriched T cells produced significantly more IFN-γ by ELISA. This finding supports the enrichment of LTA specific T cells with functional capacity to interact effectively with tumor cells (FIG. 11B).

In addition to enhanced T cell production of IFN-γ in the LTA-stimulated compared with placebo-stimulated T cell culture, it was tested whether there was a difference between the cultures in their capacity for matched tumor cell killing. Tumor cell killing was measured by incubation of 250,000 T cells with 25,000 Cell Trace Violet-labeled tumor cells for 12 hours followed by quantitation of live Cell Trace Violet-labeled cells using flow cytometry (FIG. 12A). Although a high level of baseline death was observed in cultured tumor cells in this particular experiment, which complicated data interpretation, a greater rate of death was observed in tumor cells incubated with LTA-expanded T cells compared with placebo-expanded controls, indicating increased killing capacity in the LTA-expanded group.

To validate the finding of greater killing by LTA-expanded compared with Placebo-expanded T cells, a Europium-release killing assay was carried out, in which 200,000 T cells and 10,000 tumor cells were incubated for 8 hours prior to luminescent measurement of Europium release. This assay confirmed the increased capacity of LTA-expanded T cells to kill matched patient MCC tumor cells compared with Placebo-expanded controls.

In order to further test the human in vitro vaccination response, the HLA-A2 positive MCC cell line WAGA was acquired and HLA-A2 positive MCC patients were identified using flow cytometry based HLA-typing. Using the previously described method for in vitro vaccination, the PBMC pool was enriched for LTA specific T cells by repeated pulse of antigen loaded moDCs, and then subjected to co-culture with WAGA cells, and specific killing and IFNγ release was measured compared to placebo co-culture. Flow cytometry based killing assay after 14 days of T cell enrichment showed a significant increase in HLA-matched tumor cell death over 24 hours at an E:T ration of 5:1 (FIG. 13A). In this same experiment, the LTA vaccinated PBMC pool co-cultured with HLA-matched tumor cells resulted in significantly increased IFNγ release compared to both the placebo PBMC pool and tumor cells alone (FIG. 13B). Supernatants of the PBMC pool were taken three days following each pulse of LTA or placebo loaded moDCs (T:DC 10:1) and a significant increase in IFNg was detected in the LTA vaccinated pool at each time point (FIG. 13C).

### Example 6: Vaccine-Assisted Identification of Antigen-Specific T Cell Receptors

The current vaccine was used to expand vaccine-specific T cells. As in the prior T cell expansion experiments (i.e. FIGS. 11A-11B), Merkel cell carcinoma patient blood were first isolated, generated monocyte-derived dendritic cells and serially stimulated PBMCs with either vaccine-transfected DCs or placebo-LNP-treated DCs (FIG. 14A). On d25 after the first stimulation, both vaccine expanded (LTA) and placebo-expanded (Placebo) T cells were stimulated with i) placebo-transfected DCs (Placebo-DCs); ii) tumor cells or iii) LTA-vaccine-transfected DCs (LTA-Vax-DCs). Cell hashing was used to label each culture condition, sorted for live cells using fluorescence-asssited cell sorting and performed matched single cell and TCR sequencing using the 5′ 10× Genomics platform. Following quality control, filtering, demultiplexing and alignment, clonotypes were analyzed using scRepertoire, identifying clonotypes that were specifically expanded by LTA-Vax-DCs relative to Placebo-DCs and are therefore likely to be specific to the vaccine antigen (FIG. 14B).

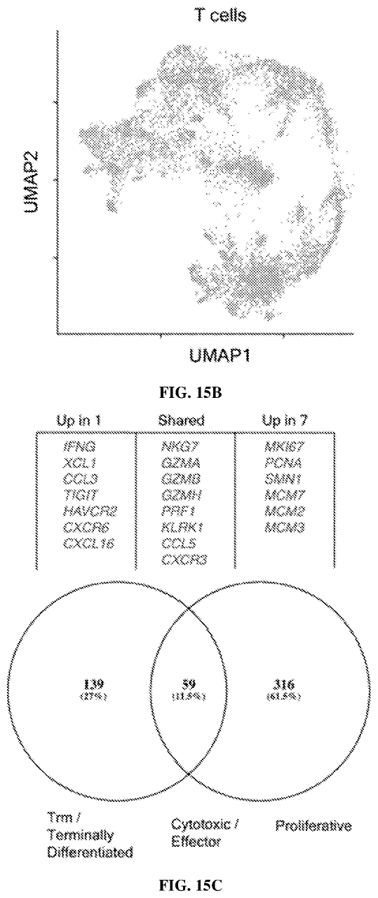

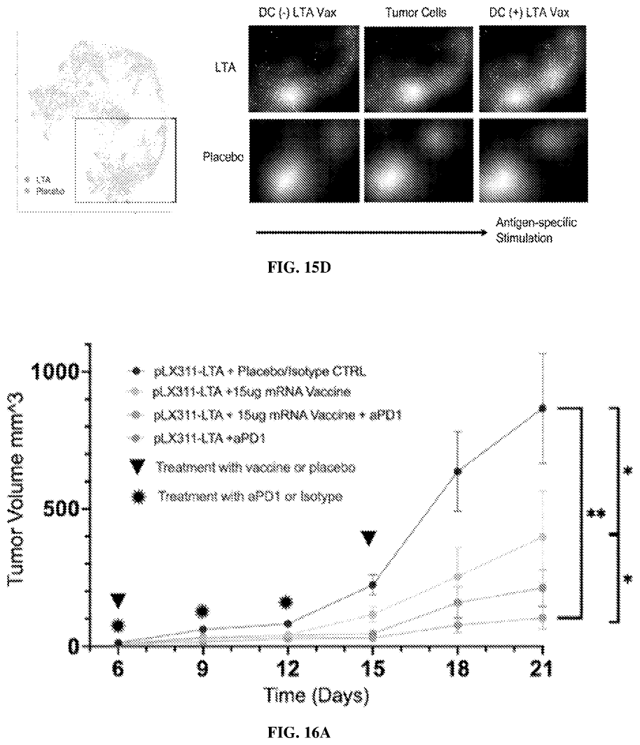

In order to understand the phenotypes of T cells expanded with the vaccine from patient blood, the transcriptional states of vaccine and placebo-expanded T cells were analyzed (FIGS. 15A-15B). Several distinct transcriptional states enriched by LTA vaccine expansion including clusters 1 and 7 (FIGS. 15A-15B) were noted. Both LTA vaccine-expanded clusters were enriched for programs of cytotoxicity (FIG. 15C). However, cluster 1 was further enriched for cytokines including IFNγ and cluster 7 was enriched for markers of proliferation. Notably, when examining overnight restimulation of LTA-expanded T cells with Placebo-DCs, patient matched tumor cells and LTA-Vax-DCs, preferential induction of proliferative states in the LTA-expanded T cells following LTA-containing stimulations (tumor cells, LTA-Vax-DCs; FIG. 15D) was observed, further confirming the expansion of antigen-specific T cells by the vaccine used in this study.

### Example 7: Combination Therapy with Anti-PD1

Standard of care for metastatic merkel cell carcinoma includes treatment with anti-PD1 antibodies. Combination therapy with LTA vaccination and anti-PD1 was tested in the B16-LTA murine model to evaluate co-therapy with current standard of care. 1 million B16-LTA cells were injected subcutaneously on the right flank on day 0, and mice were treated with LTA mRNA 15 ug IM injection on day 6 and 15, 200 ug of aPD1 antibodies by IP injection on day 6, 9, and 12, a combination of both, or volume matched placebo. Tumor growth and survival were tracked, and combination therapy resulted in increased growth suppression compared to LTA vaccine alone (FIG. 16A). Median survival was also increased in combination therapy, P=0.054 (FIG. 16B). N=10 mice per group.

### Example 8: Ex Vivo Analysis of Immune Activation Following LTA Vaccination

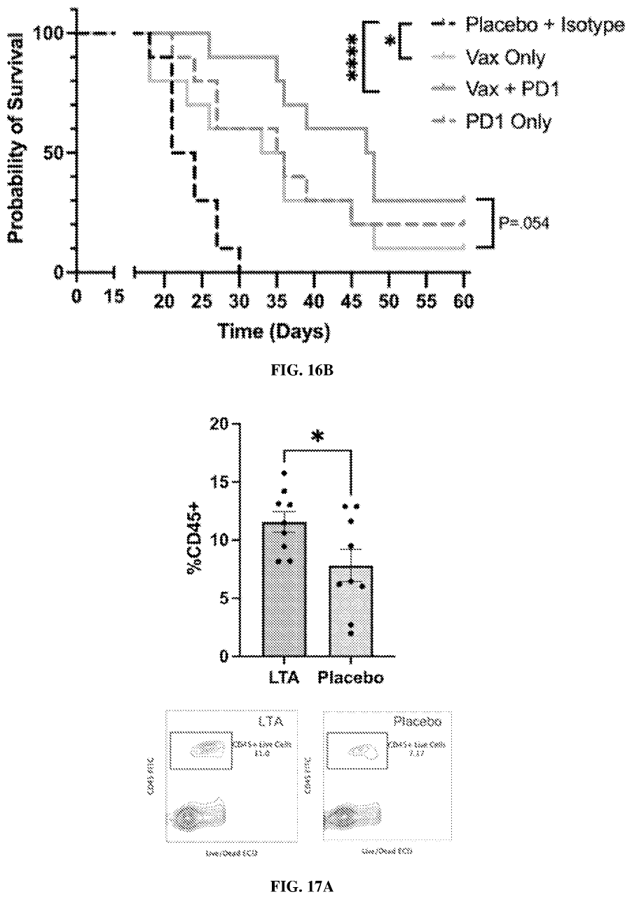

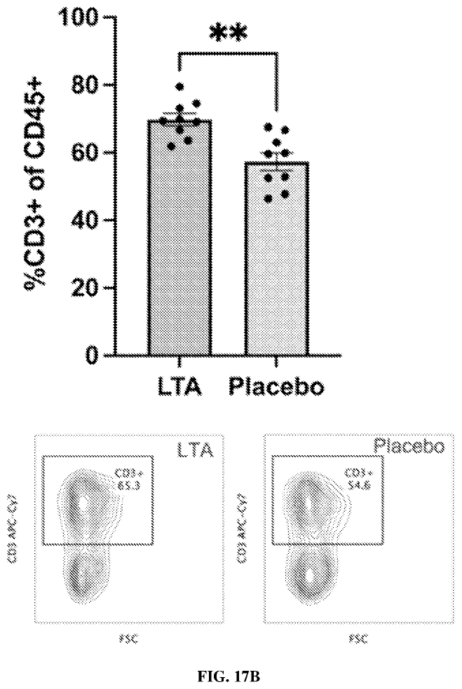

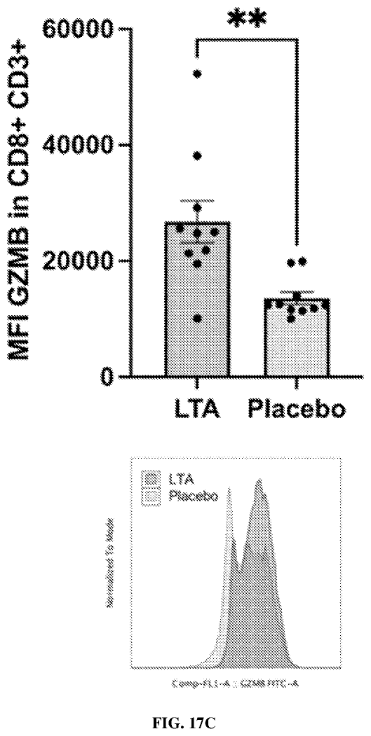

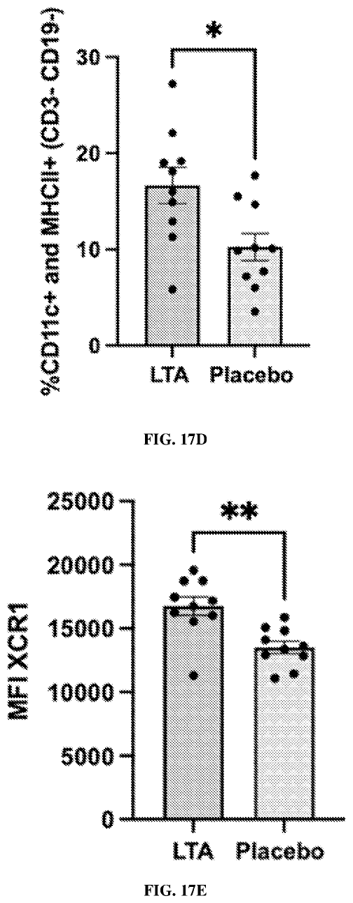

Mice bearing B16-LTA tumors were treated with 15 ug LTA mRNA vaccine or volume matched placebo on day 6 and 12, and were euthanized on day 15 (N=10 per group). The tumors were dissected and processed into single cell suspension, and were then analyzed by flow cytometry to interrogate the mechanism of vaccine mediated tumor growth suppression. Flow cytometry revealed a significant increase in total tumor immune infiltration (% CD45+ of live cells) in LTA vaccine treated vs. placebo tumors (FIG. 17A). Among these infiltrating immune cells, there was a significant increase in the proportion of T cells (% CD3+ of CD45+) in LTA vaccine treated mice vs. placebo (FIG. 17B). Among CD8+ infiltrating T cells, there was a significant increase in cytotoxic potential (MFI GZMb) in LTA vaccine treated mice vs. placebo (FIG. 17C). In the same experiment, vaccine draining lymph nodes (left inguinal) were also harvested and processed into single cell suspension and analyzed by flow cytometry. This analysis revealed a significant increase in the proportion of dendritic cells (CD11c+ and MHCII+ of CD3− and CD19−) in the LTA vaccine treated nodes vs. placebo (FIG. 17D). Among these dendritic cells, there was a significant increase in the cDC1 marker XCR1 in the LTA vaccine treated mice vs. placebo (FIG. 17E). Taken together, these results indicate that control of tumor growth in LTA vaccine treated mice is mediated by increased antigen presentation in the vaccine draining lymph node by cDC1 cells, which produce a population of antigen specific T cells with cytotoxic potential that traffic to the tumor, where they kill LTA expressing tumor cells.

### Example 9: Enhanced Efficacy of LTA Vaccine in Single Cloned B16-LTA Tumor Model

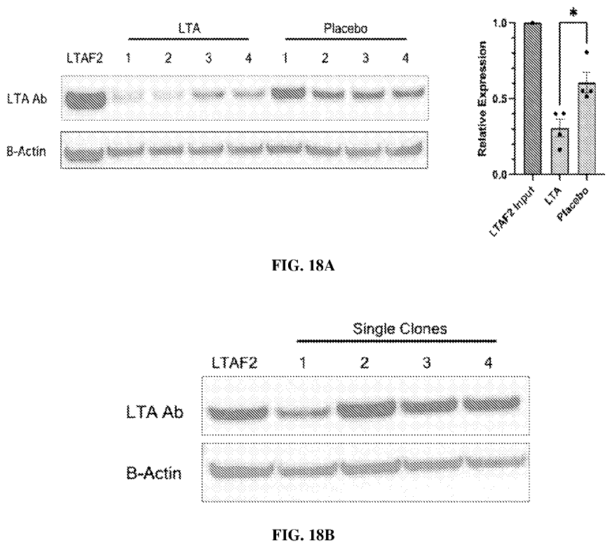

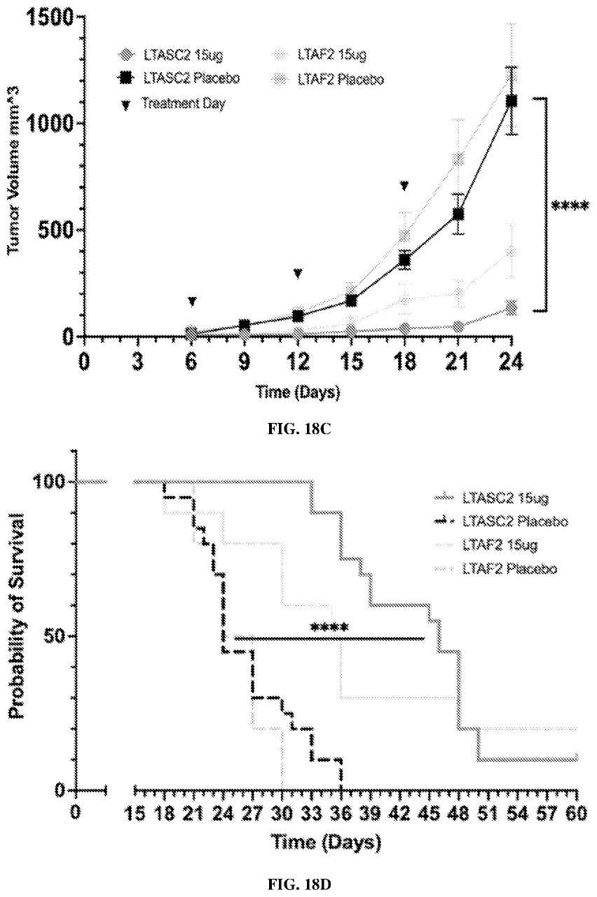

In order to interrogate the mechanism of resistance to vaccination for B16-LTA tumors that showed an initial therapeutic response, mice bearing B16-LTA tumors that were treated with either 15 ug LTA mRNA vaccine or volume matched placebo on day 6 and 15 were euthanized on day 24, and their tumors were dissected and processed for single cell suspension. The tumor cells were lysed, and LTA expression was measured using western blot. Western blot revealed a significant decrease in LTA expression among tumor cells that survived treatment with LTA mRNA vaccination compared to placebo (FIG. 18A). These findings further indicate that the vaccine leads to tumor cell killing in an antigen specific manner, but that loss of the antigen expression represents a mechanism of resistance to therapy.

To further test this hypothesis, the B16-LTA model was single cell cloned to decrease genetic diversity and diversity in LTA expression, therefore mitigating resistance by model antigen loss. Single cell clones were screened against the parental line for LTA expression by western blot, and a single clone was selected (LTASC2) for additional in vivo testing (FIG. 18B).

Comparison of the single cloned tumor model with the parental line resulted in increased growth suppression and increased survival, indicating that genetic diversity and diversity in antigen expression in the tumor was acting as a mode of vaccine resistance (FIGS. 18C-18D). C57BL/6 mice were implanted with 1 million LTASC2 tumor cells by subcutaneous injection on the right flank on day 0, and were treated with 15 ug LTA mRNA vaccine or volume matched placebo on day 6, 12, and 18, and tumor growth and survival was tracked.

### Example 10: Prophylactic LTA Vaccination Results in Complete Tumor Rejection

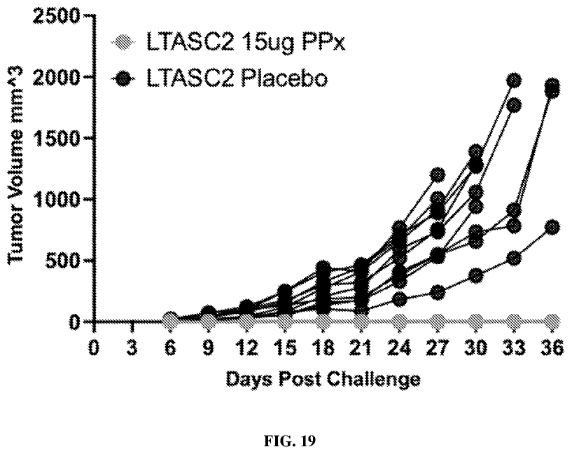

In order to test memory formation and the potential of a prophylactic use for the LTA vaccine, 12 week old female C57BL/6 mice were treated with 15 ug of LTA mRNA vaccine or volume matched placebo on day 0, 6, and 30. On day 60, 1 million B16-LTA (LTASC2) cells were injected subcutaneously on the right flank, and tumor growth and survival were tracked. Prophylactic vaccination resulted in complete rejection of 10/10 tumors, and 0/10 in the placebo group (FIG. 19). These data indicate a robust antigen specific memory formation following vaccination and the possibility of prophylactic efficacy in high risk populations.

### SUMMARY

Recent advances in mRNA vaccination for SARS-CoV-2 have demonstrated an exceptional potential for mRNA vaccines to drive effective and long-lasting immunity by T cells and other immune cell types. Furthermore, in the case of MCC and other virally driven cancers, a substantial body of evidence indicates that virally derived peptides (and particularly peptides derived from the LTA and STA of the MCC polyomavirus) have the potential to drive effective T cell responses. Therefore, an MCC mRNA vaccine has been designed to target the consensus integrated region of LTA and/or STA that was identified in multiple patient sequences. A published synthetic promoter and the signal peptide from the SARS-CoV-2 spike protein were incorporated into the design.

To validate as a vaccine candidate, an artificial LTA or STA-bearing transplantable tumor model was constructed using the B16 murine melanoma cell line, showing that it stimulates effective control of this challenging tumor model. An approach was also developed to human peripheral blood stimulations using MCC polyomavirus+ blood samples from healthy donors and MCC patients under an approved IRB protocol. This approach allows for rapidly expansion of antigen-specific T cells and for identification of their receptor sequences, enabling the development of cellular and protein-TCR-based therapeutics. These approaches are also applicable to other cancers and particularly to other virally driven cancers (e.g., HPV+ squamous cell tumors including head and neck or cervical cancers). Furthermore, the peripheral blood stimulation approach developed here will facilitate the rapid enrichment of antigen-specific T cell clonotypes, and permit the development of T cell therapeutics on a timescale that is shorter and has more flexible design and TCR search parameters than has previously been possible.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

1. An immunogenic composition comprising a nucleic acid encoding a viral antigen or an immunogenic fragment thereof, and optionally an adjuvant, wherein the viral antigen is derived from a virus that causes cancer.

2. The immunogenic composition of claim 1, wherein the viral antigen is derived from a virus selected from the group consisting of Merkel Cell Polyomavirus (MCPyV), Hepatitis B virus (HBV), Hepatitis C virus (HCV), Human papilloma virus (HPV), Kaposi's sarcoma-associated herpesvirus, Epstein-Barr virus (EBV), and human T-cell lymphotropic virus.

3. (canceled)

4. The immunogenic composition of claim 1, wherein the viral antigen is derived from a truncated form of the viral Large T Antigen (LTA) or Small T Antigen (STA) of MCPyV, or derived from E2, E5, or E6 proteins of HPV.

5. The immunogenic composition of claim 1, wherein the nucleic acid further comprises a nucleotide sequence encoding a signal peptide, optionally wherein the signal peptide is derived from a signal peptide of a full-length coronavirus spike protein.

6.-11. (canceled)

12. The immunogenic composition of claim 1, wherein the nucleic acid further comprises a 5′ untranslated region (UTR) sequence and/or a 3′ UTR sequence.

13.-15. (canceled)

16. The immunogenic composition of claim 1, wherein the nucleic acid further comprises a poly(A) tail.

17. The immunogenic composition of claim 1, wherein the viral antigen or an immunogenic fragment thereof comprises the amino acid sequence of any one of SEQ ID NOs:1-5 or 25, and a variant thereof having at least 70%, 75%, 80%, 85%, 90%, 91%, 92%, 93%, 94%, 95%, 96%, 97%, 98%, or 99% sequence identity thereto.

18. The immunogenic composition of claim 1, wherein the nucleic acid comprises the nucleotide sequence of any one of SEQ ID NOs:6-10 or 24.

19. The immunogenic composition of claim 1, wherein the nucleic acid is an mRNA.

20. A double stranded DNA sequence comprising the immunogenic composition of claim 1.

21. The double stranded DNA sequence of claim 20 further comprising (i) one or more restriction sites, (iii) promoter region, (iv) TRILINK CAP site, and/or (v) traditional KOZAK sequence.

22.-23. (canceled)

24. A pharmaceutical formulation comprising the immunogenic composition of claim 1, and one or more pharmaceutically acceptable carrier.

25. (canceled)

26. The pharmaceutical formulation of claim 24, wherein the immunogenic composition is encapsulated within and/or associated with a delivery vehicle that increases the serum half-life of the immunogenic composition as compared to the serum half-life of the same amount of the immunogenic composition alone, optionally wherein the delivery vehicle is a lipid nanoparticle and/or optionally formulated for intranasal or by intramuscular administration.

27.-30. (canceled)

31. A method of eliciting an immune response in a subject in need thereof, comprising administering to the subject an effective amount of the pharmaceutical formulation of claim 24.

32.-38. (canceled)

39. A method for enriching T cells specific for a viral or tumor antigen, comprising
optionally i) isolating T cells from a subject; and
ii) stimulating the T cells using the viral or tumor antigen or a nucleic acid encoding the same, optionally wherein the viral or tumor antigen is encoded by the nucleic acid of claim 1.

40.-52.

53. An engineered T cell receptor (TCR) comprising an alpha chain variable domain comprising the amino acid sequence of SEQ ID NO:27, 29, 31, 33, 35, 37, 39, 41, 43, or 45, or a variant thereof with at least 70% sequence identity thereto, and/or a beta chain variable domain comprising the amino acid sequence of SEQ ID NO:28, 30, 32, 34, 36, 38, 40, 42, 44, or 46, or a variant thereof with at least 70% sequence identity thereto, wherein the TCR is specific for an LTA antigen.

54.-67. (canceled)

68. A host cell engineered to express the TCR of of claim 53, optionally wherein the cell is a T cell.

69. (canceled)

70. A method of adoptive T cell therapy comprising administering a subject in need thereof an effective amount of the T cells of claim 68.

71. A method of maturing dendritic cells (DC) optionally Mo-DC, comprising
optionally i) isolating DC from a subject; and
ii) stimulating the DC using the viral or tumor antigen or a nucleic acid encoding the same, optionally wherein the viral or tumor antigen is encoded by the nucleic acid of claim 1.

72. A method of adoptive dendritic cell therapy comprising administering a subject in need thereof dendritic cells matured according to the method of claim 71.

73. (canceled)


SEQUENCE LISTING (46 sequences)

SEQ ID NO: 1 - protein, 258 aa, synthetic construct
MDLVLNRKEREALCKLLEIAPNCYGNIPLMKAAFKRSCLKHHPDKGGNPVIMMELNTLWSKFQQNIHKLRSDFSMFDEVDEAPIYGTTKFKEWWRSGGFSFGKAYEYGPNPHGTNSRSRKPSSNASRGAPSGSSPPHSQSSSSGYGSFSASQASDSQSRGPDIPPEHHEEPTSSSGSSSREETTNSGRESSTPNGTSVPRNSSRTDGTWEDLFCDESLSSPEPPSSSEEPEEPPSSRSSPRQPPSSSAEEASSSQFTD

SEQ ID NO: 2 - protein, 186 aa, synthetic construct
MDLVLNRKEREALCKLLEIAPNCYGNIPLMKAAFKRSCLKHHPDKGGNPVIMMELNTLWSKFQQNIHKLRSDFSMFDEVSTKFPWEEYGTLKDYMQSGYNARFCRGPGCMLKQLRDSKCACISCKLSRQHCSLKTLKQKNCLTWGECFCYQCFILWFGFPPTWESFDWWQKTLEETDYCLLHLHLF

SEQ ID NO: 3 - protein, 365 aa, synthetic construct
METLCQRLNVCQDKILTHYENDSTDLRDHIDYWKHMRLECAIYYKAREMGFKHINHQVVPTLAVSKNKALQAIELQLTLETIYNSQYSNEKWTLQDVSLEVYLTAPTGCIKKHGYTVEVQFDGDICNTMHYTNWTHIYICEEASVTVVEGQVDYYGLYYVHEGIRTYFVQFKDDAEKYSKNKVWEVHAGGQVILCPTSVFSSNEVSSPEIIRQHLANHPAATHTKAVALGTEETQTTIQRPRSEPDTGNPCHTTKLLHRDSVDSAPILTAFNSSHKGRINCNSNTTPIVHLKGDANTLKCLRYRFKKHCTLYTAVSSTWHWTGHNVKHKSAIVTLTYDSEWQRDQFLSQVKIPKTITVSTGFMSI

SEQ ID NO: 4 - protein, 83 aa, synthetic construct
MTNLDTASTTLLACFLLCFCVLLCVCLLIRPLLLSVSTYTSLILLVXVLWITAASAFRCFIVYIVFVYIPLFLIHTHARFLIT

SEQ ID NO: 5 - protein, 158 aa, synthetic construct
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 6 - DNA, 777 bp, synthetic construct
  source 1..777: mol_type = other DNA
atggatttagtcctaaataggaaagaaagagaggctctctgcaagcttttagagattgctcctaattgttatggcaacatccctctgatgaaagctgctttcaaaagaagctgcttaaagcatcaccctgataaagggggaaatcctgttataatgatggaattgaacaccctttggagcaaattccagcaaaatatccacaagctcagaagtgacttctctatgtttgatgaggtcgacgaggcccctatatatgggaccactaaattcaaagaatggtggagatcaggaggattcagcttcgggaaggcatacgaatatgggcccaatccacacgggaccaactcaagatccagaaagccttcctccaatgcatccaggggagcccccagtggaagctcaccaccccacagccagagctcttcctctgggtatgggtccttctcagcgtcccaggcttcagactcccagtccagaggacccgatatacctcccgaacaccatgaggaacccacctcatcctctggatccagtagcagagaggagaccaccaattcaggaagagaatccagcacacccaatggaaccagtgtacctagaaattcttccagaacggatggcacctgggaggatctcttctgcgatgaatcactttcctcccctgagcctccctcgtcctctgaggagcctgaggagcccccctcctcaagaagctcgccccggcagcccccgtcttcctctgccgaggaggcctcgtcatctcagtttacagattag

SEQ ID NO: 7 - DNA, 561 bp, synthetic construct
  source 1..561: mol_type = other DNA
atggatttagtcctaaataggaaagaaagagaggctctctgcaagcttttagagattgctcctaattgttatggcaacatccctctgatgaaagctgctttcaaaagaagctgcttaaagcatcaccctgataaagggggaaatcctgttataatgatggaattgaacaccctttggagcaaattccagcaaaatatccacaagctcagaagtgacttctctatgtttgatgaggtcagtacaaaatttccttgggaagaatatggaactttaaaggattatatgcaaagtggatataatgctagattttgcagaggtcctgggtgcatgcttaagcaacttagagattctaagtgcgcttgtattagctgtaagttgtctcgccagcattgtagtctaaaaactttaaagcaaaaaaactgtctgacgtggggagagtgtttttgctatcagtgctttattctttggtttggatttcctcctacttgggaaagttttgactggtggcaaaaaactttagaagaaactgactactgcttactgcatctgcaccttttctag

SEQ ID NO: 8 - DNA, 1098 bp, synthetic construct
  source 1..1098: mol_type = other DNA
atggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccaccccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatga

SEQ ID NO: 9 - DNA, 165 bp, synthetic construct
  source 1..165: mol_type = other DNA
atgacaaatcttgatactgcatccacaacattactggcgtgctttttgctttgcttttgtgtgcttttgtgtgtctgcctattaatacgtccgctgcttttgtctgtgtctacatacacatcattatttttaatacatacacatgcacgctttttaattacataa

SEQ ID NO: 10 - DNA, 477 bp, synthetic construct
  source 1..477: mol_type = other DNA
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 11 - DNA, 48 bp, synthetic construct
  source 1..48: mol_type = other DNA
atgttcgtgttcctggtgctgctgcccctggtgagcagccagtgcgtg

SEQ ID NO: 12 - protein, 16 aa, synthetic construct
MFVFLVLLPLVSSQCV

SEQ ID NO: 13 - DNA, 19 bp, synthetic construct
  source 1..19: mol_type = other DNA
taatacgactcactataag

SEQ ID NO: 14 - DNA, 17 bp, synthetic construct
  source 1..17: mol_type = other DNA
attaaccctcactaaag

SEQ ID NO: 15 - DNA, 18 bp, synthetic construct
  source 1..18: mol_type = other DNA
atttaggtgacactatag

SEQ ID NO: 16 - DNA, 139 bp, synthetic construct
  source 1..139: mol_type = other DNA
acccaagctggctagcgtttaaacttaagcttggtaccgcttgtctcgctccggggaacgctcggaaactcccggccgccgccacccgcgtctgttctgttacacaagggaagaaaagccgctgccgcactccgagtgt

SEQ ID NO: 17 - DNA, 106 bp, synthetic construct
  source 1..106: mol_type = other DNA
caacaacaacaacaacaacaacaacaacaacaacaacaacaacaacaacaacaacaacaacaacaagggcattcctgaagctgacagcattcgggccgaggccacc

SEQ ID NO: 18 - DNA, 106 bp, synthetic construct
  source 1..106: mol_type = other DNA
agaccagttcttagccatcaagcagagactctgaagccagactacctgggtcccaatcttgggcttggtatttcctcgctgtgtgactctggactgcgccgccacc

SEQ ID NO: 19 - DNA, 195 bp, synthetic construct
  source 1..195: mol_type = other DNA
ggggctcctagcctccgaggcctctggaaaaaaaagagagaggcctctgaggcttaagaggcttaattagcaaaaaaggcagtatctaagggcagatcccaagggcgggaaactgcagtataaaaaccactccttagtgaggtagctcatttgctcctctgctctttctgcaaactccttctgcatatagacaag

SEQ ID NO: 20 - DNA, 111 bp, synthetic construct
  source 1..111: mol_type = other DNA
gctggagcctcggtggcctagcttcttgccccttgggcctccccccagcccctcctccccttcctgcacccgtacccccgtggtctttgaataaagtctgagtgggcggca

SEQ ID NO: 21 - protein, 8 aa, synthetic construct
DYKDDDDK

SEQ ID NO: 22 - protein, 12 aa, synthetic construct
PDRVRAVSHWSS

SEQ ID NO: 23 - protein, 8 aa, synthetic construct
WSHPQFEK

SEQ ID NO: 24 - DNA, 1221 bp, synthetic construct
  source 1..1221: mol_type = other DNA
gagctcacgcgttaatacgactcactataagacccaagctggctagcgtttaaacttaagcttggtaccgcttgtctcgctccggggaacgctcggaaactcccggccgccgccacccgcgtctgttctgttacacaagggaagaaaagccgctgccgcactccgagtgtgccaccatgttcgtgttcctggtgctgctgcccctggtgagcagccagtgcgtggatttagtcctaaataggaaagaaagagaggctctctgcaagcttttagagattgctcctaattgttatggcaacatccctctgatgaaagctgctttcaaaagaagctgcttaaagcatcaccctgataaagggggaaatcctgttataatgatggaattgaacaccctttggagcaaattccagcaaaatatccacaagctcagaagtgacttctctatgtttgatgaggtcgacgaggcccctatatatgggaccactaaattcaaagaatggtggagatcaggaggattcagcttcgggaaggcatacgaatatgggcccaatccacacgggaccaactcaagatccagaaagccttcctccaatgcatccaggggagcccccagtggaagctcaccaccccacagccagagctcttcctctgggtatgggtccttctcagcgtcccaggcttcagactcccagtccagaggacccgatatacctcccgaacaccatgaggaacccacctcatcctctggatccagtagcagagaggagaccaccaattcaggaagagaatccagcacacccaatggaaccagtgtacctagaaattcttccagaacggatggcacctgggaggatctcttctgcgatgaatcactttcctcccctgagcctccctcgtcctctgaggagcctgaggagcccccctcctcaagaagctcgccccggcagcccccgtcttcctctgccgaggaggcctcgtcatctcagtttacagattaggctggagcctcggtggcctagcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctagagagctc

SEQ ID NO: 25 - protein, 273 aa, synthetic construct
MFVFLVLLPLVSSQCVDLVLNRKEREALCKLLEIAPNCYGNIPLMKAAFKRSCLKHHPDKGGNPVIMMELNTLWSKFQQNIHKLRSDFSMFDEVDEAPIYGTTKFKEWWRSGGFSFGKAYEYGPNPHGTNSRSRKPSSNASRGAPSGSSPPHSQSSSSGYGSFSASQASDSQSRGPDIPPEHHEEPTSSSGSSSREETTNSGRESSTPNGTSVPRNSSRTDGTWEDLFCDESLSSPEPPSSSEEPEEPPSSRSSPRQPPSSSAEEASSSQFTD

SEQ ID NO: 26 - DNA, 807 bp, synthetic construct
  source 1..807: mol_type = other DNA
gagctcgctagcgccaccatggatttagtcctaaataggaaagaaagagaggctctctgcaagcttttagagattgctcctaattgttatggcaacatccctctgatgaaagctgctttcaaaagaagctgcttaaagcatcaccctgataaagggggaaatcctgttataatgatggaattgaacaccctttggagcaaattccagcaaaatatccacaagctcagaagtgacttctctatgtttgatgaggtcgacgaggcccctatatatgggaccactaaattcaaagaatggtggagatcaggaggattcagcttcgggaaggcatacgaatatgggcccaatccacacgggaccaactcaagatccagaaagccttcctccaatgcatccaggggagcccccagtggaagctcaccaccccacagccagagctcttcctctgggtatgggtccttctcagcgtcccaggcttcagactcccagtccagaggacccgatatacctcccgaacaccatgaggaacccacctcatcctctggatccagtagcagagaggagaccaccaattcaggaagagaatccagcacacccaatggaaccagtgtacctagaaattcttccagaacggatggcacctgggaggatctcttctgcgatgaatcactttcctcccctgagcctccctcgtcctctgaggagcctgaggagcccccctcctcaagaagctcgccccggcagcccccgtcttcctctgccgaggaggcctcgtcatctcagtttacagattagacgcgtgagctc

SEQ ID NO: 27 - protein, 14 aa, synthetic construct
CAFMRGGSQGNLIF

SEQ ID NO: 28 - protein, 13 aa, synthetic construct
CASTPDTSYEQYF

SEQ ID NO: 29 - protein, 15 aa, synthetic construct
CAFMRGTGANSKLTF

SEQ ID NO: 30 - protein, 13 aa, synthetic construct
CASSTPGPYEQYF

SEQ ID NO: 31 - protein, 14 aa, synthetic construct
CAGLTNFGNEKLTF

SEQ ID NO: 32 - protein, 15 aa, synthetic construct
CASSPPRDTAAEAFF

SEQ ID NO: 33 - protein, 14 aa, synthetic construct
CALSVPDSNYQLIW

SEQ ID NO: 34 - protein, 14 aa, synthetic construct
CASSFGGGNQPQHF

SEQ ID NO: 35 - protein, 13 aa, synthetic construct
CALSYTGGFKTIF

SEQ ID NO: 36 - protein, 15 aa, synthetic construct
CASSPPDSSNQPQHF

SEQ ID NO: 37 - protein, 11 aa, synthetic construct
CATNRDDKIIF

SEQ ID NO: 38 - protein, 14 aa, synthetic construct
CASSLGTGEVEQYF

SEQ ID NO: 39 - protein, 12 aa, synthetic construct
CAVDGTASKLTF

SEQ ID NO: 40 - protein, 13 aa, synthetic construct
CSVGGQNTGELFF

SEQ ID NO: 41 - protein, 13 aa, synthetic construct
CAVFSGGYNKLIF

SEQ ID NO: 42 - protein, 15 aa, synthetic construct
CASSSAGVGADTQYF

SEQ ID NO: 43 - protein, 13 aa, synthetic construct
CAVGGGGYQKVTF

SEQ ID NO: 44 - protein, 13 aa, synthetic construct
CASNGGPNYGYTF

SEQ ID NO: 45 - protein, 13 aa, synthetic construct
CAVFSGGYNKLIF

SEQ ID NO: 46 - protein, 15 aa, synthetic construct
CASSSAGVGADTQYF